(19) United States
(12) Patent Application Publication
(10) Pub. No.: US 2008/0248086 A1
(43) Pub. Date: Oct. 9, 2008

(54) CURABLE THERAPEUTIC IMPLANT COMPOSITION

(75) Inventor: Soheil Asgari, Wiesbaden (DE)

Correspondence Address: DORSEY & WHITNEY LLP;INTELLECTUAL PROPERTY DEPARTMENT, 250 PARK AVENUE, NEW YORK, NY, 10177, US

(73) Assignee: CINVENTION AG, Wiesbaden (DE)

(21) Appl. No.: 12/062,689

(22) Filed: Apr. 4, 2008

Related U.S. Application Data
(60) Provisional application No. 60/910,455, filed on Apr. 5, 2007.

(51) Int. Cl. A61F 2/04

(52) U.S. Cl. 424/426

(57) ABSTRACT

The exemplary embodiments of the present invention relates to a curable therapeutic implant composition for use in the filling of a cavity in a living organism, comprising particles of a metallic material, and a curable matrix-forming, non-particulate material, wherein at least one of the metallic material or the matrix-forming material is at least partially degradable in-vivo. Furthermore, the exemplary embodiments of the present invention relate to methods of filling a cavity in a living organism with the use of the curable implant composition.

### CROSS-REFERENCE TO RELATED APPLICATION(S)

The present invention claims priority of U.S. provisional application Ser. No. 60/910,455 filed Apr. 5, 2007, the entire disclosure of which is incorporated herein by reference.

### FIELD OF THE PRESENT INVENTION

The present invention relates to a curable therapeutic implant composition for use in the filling of a cavity in a living organism, e.g., comprising particles of a metallic material, and a curable matrix-forming, non-particulate material, wherein at least one of the metallic material or the matrix-forming material is at least partially degradable in-vivo. Furthermore, the present invention, also relates to methods of filling a cavity in a living organism with the use of the curable implant composition.

### BACKGROUND INFORMATION

Implants are increasingly used in surgical, orthopedic, dental and other related applications such as tissue engineering. However, the conventional implant technology is generally focused on improving implants by making them combination products, e.g., combining drugs or therapeutically active agents with implants, such as drug-eluting coatings, or by incorporating those agents into the implant body. Other research and development is focused on increasing the contact surface between the tissue and implant surface. In certain treatment, bone defects can be treated by using cements or cement-like materials comprising ceramic materials or polymer ceramic composites. In addition, the treatment of bone defects can involve the implantation of an autograft, an allograft, or a xenograft in the defected site. Biological implants and grafts may suffer of many issues such as shortage of donor tissue, infectious contamination by bacteria or virus and others. A synthetic implant may comprise, in those cases, potential alternatives.

Due to biomechanical and physiologic preferences, an implant material should have a certain mechanical strength or elasticity to be incorporated into the target tissue and anatomic region, on the other hand desired functions such as degradability or incorporating beneficial agents such as pharmacologically or therapeutically active agents are mostly contradictory to the foregoing. For example, a range of bone grafting materials are established in clinical use, such as demineralized human bone matrix, bovine collagen mineral composites and processed coralline hydroxyapatite, calcium sulphate scaffolds, bioactive glass scaffolds and calcium phosphate scaffolds. Such orthopedic scaffolds can be used as both temporary and permanent conduits for bone. Those materials may also be used to facilitate and direct the growth of bone or cartilage tissue across sites of fractures or to re-grow them in defective, damaged or infected bone.

The provision of appropriate scaffolds can also consider the structure of bone that has to be treated. Cortical and cancellous bone can be structurally different, although the material composition may be very similar. Cancellous bone can comprise a thin interstitium lattice interconnected by pores of about 500-600 micron width with a spongy and open-spaced structure, whereby the interstitium can be substituted by a scaffolding material. Cortical bone comprises neurovascular “Haversian” canals of about 50-100 micron width within a hard or compact interstitium. A suitable scaffold may allow at least osteoconduction or osteoinduction. Osteoinductive materials can actively trigger and facilitate bone growth, for example, by recruiting and promoting the differentiation of mesenchymal stem cells into osteoblasts. Osteoconductive materials may induce bone to grow in areas where it would not normally grow, also called “ectopic” bone growth, usually by biochemical and/or physical processes. Osteogenic materials contain cells that can form bone or can differentiate into osteoblasts.

When using degradable scaffolds it can be desirable, that the degradation rate approximately matches to the re-growth or repair rate of the tissue treated. Typical biodegradation rates for maintaining the structure or structural integrity of a scaffold can be, for example, about 4-10 weeks for cartilage repair and about 3-8 weeks for bone repair. The mechanical requirements of the scaffolds can be highly dependant on the type of tissue being replaced, for example cortical bone has a Young Modulus of 15-30 GPa, whereby cancellous (or spongy, trabecular) bone generally has a Young Modulus of 0.01-2 GPa. Cartilage has a Young Modulus of less than about 0.001 GPa. It can be desirable that the materials used for a scaffold in any particular case should reflect this as far as possible.

For example, it may be desirable to have an implant material that allows osseointegration. Known implants either provide a rough surface, usually made from metals such as titanium, titanium alloys, stainless steel or cobalt chromium, or sometimes a porous surface. When using such materials, the osseointegration is typically only a mechanical integration that typically is poor or incomplete. Other reasons of incomplete integration are due to weak bone of the patient, for example, due to cancerous diseases or osteoporosis. However, a rough or porous surface may usually be applied to dense metal implants, for example by thermal spraying, surface abrasion, pitting, or other methods. Other solutions may provide a coating of hydroxyapatite, that usually is coated onto the surface of such conventional implants. It is a known issue that the adhesion of hydroxyapatite is not very strong and depending on the physiologic fluids present, in case of inflammation for example comprising acidic pH, the loosening of the hydroxyapatite occurs regularly.

Other reasons for implant failure can be that dense implants are embedded non-physiologically into the surrounding tissue, inherently with suboptimal biomechanical integration into the part of the body or tissue, for example frequently causing micro fractures or, because of insufficient osseointegration, micro movements. One exemplary effect of implant failure, regardless of the real cause, can be a peri-implantitis, acute, subacute or chronic inflammation that continuously affects or opposes the intended implant function. Specifically in critical implant regions, such as dental implants, the biologic environment and physiologic conditions is a complicating factor with a higher risk of infections due to the microbial, bacterial or fungi flora. Typical effects that may be caused by peri-implantitis are inflammation of mucosa, loss of attached gingival, exposure of a cervical portion of the implant and loss of the surrounding bone and functional implant failures. Even in dental treatments with extraction of a tooth an open wound is caused that might be contaminated by bacteria. A further significant issue is that the absence of the tooth induces spontaneously alveolar bone remodeling with resulting atrophy. Atrophy may subsequently cause more complex complications for reconstruction.

U.S. Patent Publication No. 2005/249773 describes a degradable implant composition based on biocompatible ceramics and minerals, biocompatible glasses, and biocompatible polymers, and the use thereof for e.g. in-situ replicating a bone defect, or shaping an implant in a mold ex-situ. European Patent Publication No. 1344538 describes a method to produce and a porous biodegradable implant based on biocompatible ceramics, biocompatible glasses, biocompatible polymers, and combinations thereof.

There are several disadvantages related to the use of ceramic materials in curable implant compositions. For example, one of the disadvantages of using hydroxyl apatite crystalline forms in such materials may be its lack of microporosity and mechanical stability. For adequate bone in-growth, it is conventionally known that a porosity of, e.g., at least about 100 μm or even more should be used that generally may not be obtained by ceramic or crystalline forms of hydroxyl apatite. Another exemplary drawback can be the inferior mechanical stability of hydroxylapatite that is brittle, and thus typically not suitable for stem replacement in implants. Conventional solutions with only coating a metal implant surface with hydroxyl apatite can be prone to fatigue-related destruction of the coating.

The exemplary application of hydroxyl apatite based cements can further comprise a significant issue of mechanical stability and stress shielding as the formation of natural bone tissue is a physiologic process over time whereby during the engraftment phase the materials applied as hydroxyl apatite-based cements do not provide a sufficient biomechanical stability unless the engraftment process is completed. The use of polymers may also comprise constraints due to the fact that polymers are prone to suffer from creep and fatigue. Although acrylate-based polymers are known in the art as an ingredient of cements or filling materials, such materials are not capable to promote bone formation. To the contrary, such materials generally induce the formation of fibrous membranes separating the implant material from the host bone tissue.

Metals in curable, cement-like materials are usually favorable in terms of toughness, ductility and fatigue resistance. On the other hand they are known to be stiffer than natural bone, resulting in stress shielding. The phenomenon of stress shielding is well known and based on the effect that the implant material bears more of mechanical loads if it is stiffer than the surrounding tissue. This can result in a “shielding” of the natural bone tissue from the mechanical load triggering the resorption processes of bone. Other ceramic implant materials are known to be prone to micro cracks particularly when impulsive forces occur.

It is also known that polymers or, e.g., acrylate based cements should be mixed with radiopaque compounds, such as barium or iodine salts, to make them radiopaque.

### SUMMARY OF EXEMPLARY EMBODIMENTS OF PRESENT INVENTION

One exemplary object of the present invention is to provide a class of implant materials for orthopedic, surgical, dental and traumatologic implants, particularly implant materials for substituting or repairing, e.g., bone defects, producible and formable in-situ and/or in-vivo, or ex-situ and/or ex-vivo.

A further exemplary object of the present invention is to provide a class of implant materials that can be used as a cement for orthopedic, surgical, dental and traumatologic implants. Preferably, the exemplary implant materials may provide an adjustable, accurate biodegradation in-vivo, and may be tailored to provide additional functions, such as incorporating or releasing beneficial agents.

According to another exemplary embodiment of the present invention, a curable therapeutic implant composition for use in the filling of a cavity in a living organism can be provided, comprising particles of a metallic material, and a curable matrix-forming, non-particulate material, whereas at least one of the metallic material or the matrix-forming material can be at least partially degradable in-vivo.

The composition may be used as a cement for filling of a cavity in a living organism e.g. for repairing a bone, tooth or cartilage defect in a living organism in-situ, e.g., in-vivo. Furthermore, the exemplary implant composition may be used as a cement for fixation of implants or bone, or for repairing a bone fissure in a living organism in-vivo. Alternatively or in addition, the composition may be used for producing a shaped implant for repairing a bone or cartilage defect in a living organism ex-vivo. For example, the composition may be used for producing a tissue scaffold, an implantable fracture fixation device such as plates, screws and rods, a dental implant, an orthopedic implant, a traumatologic implant, or a surgical implant.

In yet another exemplary embodiment of the present invention, the metallic material particles include at least one of a metal or a metal alloy. According to a further exemplary embodiment of the present invention, the metallic material particles can be completely degradable in-vivo.

According to an alternative exemplary embodiment of the present invention, the metallic material particles are substantially not degradable in-vivo. According to a further exemplary embodiment of the present invention, the composition can include particles of metallic material selected from a biocorrosive alloy, or a mixture of at least one first metallic material and at least one second metallic material, the first metallic material being more electronegative than the second metallic material, such that the first and second metallic material particles form a local cell at their contact surfaces. In such exemplary embodiment, the less noble metal is preferentially degraded in-vivo.

According to a first aspect, the composition as described herein includes an organic material as the curable matrix-forming, non-particulate material, preferably a polymer-solvent system. The polymer-solvent system can comprise a mixture of at least one polymer and at least one solvent or plasticizer. Typically, such polymer-solvent based compositions are curable or may be hardened by extraction of the solvent or plasticizer, such as diffusion of the solvent or plasticizer into an aqueous medium ex-vivo, or into body fluids in-vivo. Alternatively or in addition, the extraction may involve removal of the solvent or plasticizer by e.g. heat or pressure treatments.

According to yet another exemplary embodiment of the present invention, the curable matrix-forming, non-particulate material in an exemplary embodiment can be an organic material comprising at least one polymerizable or crosslinkable monomer, for example a monofunctional monomer or a polyfunctional monomer, preferably (meth)acrylates. In addition, mixtures of monofunctional and polyfunctional monomers may be used.

For curing or hardening the composition of such embodiments, the composition can additionally comprise a polymerization catalyst, an initiator, or an accelerator.

According to still a further exemplary embodiment of the present invention, the compositions can involve a two component system, whereas the composition may be divided into two parts, and the both parts are to be mixed at the time of use.

According to another exemplary embodiment of the present invention, the curable matrix-forming, non-particulate material of an exemplary embodiment can include precursor compounds of an inorganic-organic hybrid material, processible by sol-gel processing. The sol-gel-processing can be either a hydrolytic or non-hydrolytic sol-gel processing. The precursor compounds processible by sol-gel processing can include at least one metal alkoxide. In such embodiments, the composition may further comprise at least one suitable crosslinking agent.

In a further exemplary embodiment, the metal alkoxide includes a hydrolytically condensable, organically modified trialkoxysilane which contains free-radically polymerizable acrylate or methacrylate groups or cyclic groups capable of ring opening polymerization.

In addition, the exemplary curable matrix-forming material may include a combination of any of the above described embodiments of the present invention.

According to still another exemplary embodiment of the present invention, the composition can be curable by drying, solvent extraction, radiation, such as visible light, UV or IR radiation, heat, polymerization or chemical crosslinking.

Also, the exemplary compositions of exemplary embodiments may further comprise conventional additives such as a crosslinker, a coupling agent, a plasticizer, a solvent, a filler, a pigment, or a beneficial agent, which may optionally be configured to be released in-vivo from the final implant.

According to another exemplary embodiment of the present invention, a method of filling a cavity in a living organism is provided, which in the exemplary embodiment can comprise the filling of the cavity with the implant composition as described herein in-vivo, and subsequently curing the composition.

According to another exemplary embodiment of the present invention, the method can comprise shaping the composition as described herein ex-vivo into a desired shape for filling the cavity, for example in a mold, curing the composition; and subsequently implanting the cured composition into the cavity in the living organism. For example, the cavity may be a defect or wound in a bone, or tooth or cartilage of a living organism.

A further exemplary embodiment of the present invention may provide implants made from the compositions as described herein, preferably orthopedic, surgical, dental and traumatologic implants. According to another exemplary embodiment of the present invention, a class of implants can be provided, whereby the mechanical, chemical, biological and physical properties such as electrical conductivity, optical or other suitable properties can be tailored appropriately to the intended use.

In one further exemplary embodiment of the present invention, a composition can be provided for producing an implant in-vivo or ex-vivo whereby the degradation rate can mostly independent of the mechanical and other biological properties tailored. For example, an exemplary curable compositions and implants producible thereof can be tailored to have osteoconductive or osteoinductive, or combined properties.

In still another exemplary embodiment of the present invention, the scaffold or implant producible from the compositions as described herein can be provided may comprise rationally designed structures to allow engraftment, ingrowth, induction or conduction or any combination thereof.

### DETAILED DESCRIPTION OF EXEMPLARY EMBODIMENTS

The terms “active ingredient”, “active agent” or “beneficial agent” as used herein can include but in no way limited to any material or substance which may be used to add a function to the implantable medical device. Examples of such active ingredients can include biologically, therapeutically or pharmacologically active agents such as drugs or medicaments, diagnostic agents such as markers, or absorptive agents. The exemplary active ingredients may be a part of the first or second particles, such as incorporated into the implant or being coated on at least a part of the implant. Biologically or therapeutically active agents can comprise substances being capable of providing a direct or indirect therapeutic, physiologic and/or pharmacologic effect in a human or animal organism. A therapeutically active agent may include a drug, pro-drug or even a targeting group or a drug comprising a targeting group. An “active ingredient” according to an exemplary embodiment of the present invention may further include a material or substance which may be activated physically, e.g. by radiation, or chemically, e.g., by metabolic processes.

The term “biodegradable” as used herein can include but in no way limited to any material which can be removed in-vivo, e.g., by biocorrosion or biodegradation. Thus, any material, e.g., a metal or organic polymer that can be degraded, absorbed, metabolized, or which is resorbable in the human or animal body may be used either for a biodegradable metallic layer or as a biodegradable template in the embodiments of the present invention. In addition, as used in this description, the terms “biodegradable”, “bioabsorbable”, “resorbable”, and “biocorrodible” are can encompass but in no way limited to materials that are broken down and may be gradually absorbed or eliminated by the body in-vivo, regardless whether these processes are due to hydrolysis, metabolic processes, bulk or surface erosion.

The term (meth)acrylate as used herein can include but in no way limited to acrylates and methacrylates, which may be substituted or not, unless specified otherwise.

The following description makes reference to certain specific details of particular exemplary embodiments in order to provide a thorough understanding of the exemplary embodiments of the present invention. However, each and every specific detail needs not to be employed to practice the exemplary embodiments of the present invention, and indeed, numerous variations can be employed which are within the scope of the exemplary embodiments of the present invention.

In certain exemplary embodiments of the present invention, a curable composition may be provided which can be used, e.g., as a cement for filling a cavity in a living organism in-situ, or as a cement-like implant or bone graft molded ex-situ for direct application or implantation. Thus, certain options can be used for using the compositions. One exemplary option can be to pour out a bone defect or area of replacement to obtain the preferred physiologic and/or anatomic shape of the material by using a moldable composition directly in-vivo. Another exemplary option is to mold the composition ex-situ, e.g., outside the body of the living organism, e.g., a human, either with a mold or replica of the defective area, or in other degree of freedom.

With the compositions of the exemplary embodiments of the present invention, a material can be provided which after hardening or curing is partially or completely degradable in-vivo. By suitable selection of the metallic material and/or the curable matrix-forming material, wherein at least one of these materials is biodegradable, it is possible to fill a cavity in a living organism such as a bone defect with a biocompatible material that exhibits the desired mechanical properties directly after implantation. Furthermore, it is possible to select the materials used and their combination in the composition such that due to an at least partial degradation of the cured material, whereby the degradation rate can be controlled, a porous structure is formed in the body which allows a stepwise ingrowth of surrounding tissue and an incorporation of the implant material over time, thus promoting healing of the wound or cavity filled. Thus, with the compositions of exemplary embodiments, a temporarily tailorable variation of the properties of the implanted material depending on the progress of healing of the defect may be provided. Hence, before biodegradation starts, the implanted material can allow to mechanically resist biomechanical loads while in the mid- and long-term at least a part of the implant material will be replaced during degradation by ingrowing tissue that increases the flexibility and biomechanical properties by substituted natural tissue. Another exemplary advantage is that the exemplary embodiment of the present invention, allows to additionally functionalize the implant material obtained from the present inventions compositions, for example by adding functional compounds, such as radiopaque particles such as biocompatible metals, or to tailor specifically the mechanical properties such as flexibility by introducing e.g. fibers. Moreover, the addition of e.g. anti-microbial agents, such as silver or copper to the composition allow to increase the anti-infective properties of the implant.

In an exemplary embodiment, the composition can be applied to the defective area for replacement of bone. For example, the presence of degradable metallic particles then allows to form an interconnected network of the residual materials by degradation of the metal in situ. Another option can be to prepare the moldable composition based on a degradable matrix material such as a gel or a biodegradable polymer or a mixture of both, then the particles used must not necessarily be degradable particles. The exemplary material may be solidified or hardened in-situ. Optionally, the exemplary composition can be formed to any desired shape ex-situ.

A further exemplary embodiment can use a degradable polymer, pre-polymer or any mixture thereof that is dissolved in a biocompatible solution and can be hardened in situ. Then, in addition, the used metal-based particles must not be necessarily degradable.

According to another exemplary embodiment of the present invention, a curable therapeutic implant composition for use in the filling of a cavity in a living organism is provided, comprising particles of a metallic material, and a curable matrix-forming, non-particulate material, wherein at least one of the metallic material or the matrix-forming material is at least partially degradable in-vivo.

The exemplary composition may be used as a cement for filling of a cavity in a living organism e.g. for repairing a bone, tooth or cartilage defect in a living organism in-situ, i.e. in-vivo. Furthermore, the implant composition may be used as a cement for fixation of implants or bone, or for repairing a bone fissure in a living organism in-vivo. Alternatively or in addition, the composition may be used for producing a shaped implant for repairing a bone or cartilage defect in a living organism ex-vivo. For example, the exemplary composition may be used for producing a tissue scaffold, an implantable fracture fixation device such as plates, screws and rods, a dental implant, an orthopedic implant, a traumatologic implant, or a surgical implant.

In a further exemplary embodiment, at least one of the materials used, e.g., the metallic particles or the cured matrix-forming material is degradable in-vivo. Preferably, the exemplary composition can be adapted to provide, after hardening or curing and degradation of the first degradable constituents, an open, interconnected network of porous or capillary or combined compartments, whereby degradation can take place partially or completely in situ or ex-situ or, respectively, in the living body or during preparation or manufacturing of the implant, or any combination thereof. These exemplary compartments can be constituted by the non-degradable second materials that demarcate the interconnected network of compartments.

Exemplary Metallic Material Particles

In another exemplary embodiment of the present invention, the metallic material particles include at least one of a metal or a metal alloy, e.g. selected from main group metals of the periodic system, transition metals such as copper, gold, silver, titanium, zirconium, hafnium, vanadium, niobium, tantalum, chromium, molybdenum, tungsten, manganese, rhenium, iron, cobalt, nickel, ruthenium, rhodium, palladium, osmium, iridium or platinum, or from rare earth metals, and alloys or any mixtures thereof.

The exemplary metallic particles used in certain exemplary embodiments can be, without excluding others, e.g., —iron, cobalt, nickel, manganese or mixtures thereof, e.g., iron-platinum-mixtures, or as an example for magnetic metal oxides iron oxides and ferrites. For example, for the exemplary compositions with magnetic or signaling properties in general, magnetic metals or alloys like ferrites, e.g. gamma-iron oxide, magnetite or ferrites of Co, Ni, Mn may be used. Examples are described in International Patent Publications WO83/03920, WO83/01738, WO85/02772, WO88/00060, WO89/03675 and WO90/01295, in U.S. Pat. Nos. 4,452,773, 4,675,173 and 4,770,183. In certain exemplary embodiments, it can be preferred to select the particles from shape memory alloys, such as nickel titanium, nitinol, copper-zinc-aluminium, copper-aluminum-nickel, and the like.

In other exemplary embodiments, the particles are selected from biodegradable metals or alloys, metallic particle mixtures or metal composites. Suitable biodegradable metals can include, e.g., metals, or metal alloys, including alkaline or alkaline earth metals, Fe, Zn or Al, such as Mg, Fe or Zn, and optionally alloyed with or combined with other particles selected from Mn, Co, Ni, Cr, Cu, Cd, Pb, Sn, Th, Zr, Ag, Au, Pd, Pt, Si, Ca, Li, Al, Zn and/or Fe.

In addition, metal oxides, nitrides carbides, ceramic materials etc. may be added in certain embodiments, e.g., alkaline earth metal oxides or hydroxides such as magnesium oxide, magnesium hydroxide, calcium oxide, and calcium hydroxide or mixtures thereof.

In further exemplary embodiments, the biodegradable metal particles may be selected from biodegradable or biocorrosive metals or alloys based on at least one of magnesium or zinc, or an alloy possibly comprising at least one of Mg, Ca, Fe, Zn, Al, W, Ln, Si, or Y, such as e.g. a Mg—Ca alloy, Mg—Zn alloy, Mg—Al—Zn alloy, e.g. commercially available AZ91D, LAE442, AE21.

Furthermore, the metallic particles may be substantially completely or at least partially degradable in-vivo. Examples for suitable biodegradable alloys comprise e.g., magnesium alloys comprising more than about 90% of Mg, about 4-5% of Y, and about 1.5-4% of other rare earth metals such as neodymium and optionally minor amounts of Zr, wherein the components are selected to add up to about 100%; or biocorrosive alloys comprising as a major component tungsten, rhenium, osmium or molybdenum, for example alloyed with cerium, an actinide, iron, tantalum, platinum, gold, gadolinium, yttrium or scandium.

In a further exemplary embodiment of the present invention, the degradable metallic material particles may comprise a metal alloy of (i) about 10-98 wt.-%, such as about 35-75 wt.-% of Mg, and about 0-70 wt.-%, such as about 30-40% of Li and about 0-12 wt.-% of other metals, or (ii) about 60-99 wt.-% of Fe, about 0.05-6 wt.-% Cr, about 0.05-7 wt.-% Ni and up to about 10 wt.-% of other metals; or (iii) about 60-96 wt.-% Fe, about 1-10 wt.-% Cr, about 0.05-3 wt.-% Ni and about 0-15 wt.-% of other metals, wherein the individual weight ranges are selected to add up to about 100 wt.-% in total for each alloy.

In such exemplary embodiments, the metallic particles can be mainly degraded to produce hydroxyl apatite and H₂-gas within the living body in presence of physiologic fluids. Hydroxyl apatite may the induce or guide ingrowths of natural surrounding tissue into the residual implant structure. This exemplary property of the exemplary embodiment of the composition material can be advantageous for implants with a temporary function, and with sufficient mechanical stability compared to bioceramics or hydroxyl apatite or polymers alone.

According to another exemplary embodiments of the present invention, by alloying the aforesaid metals it is, e.g., possible to tune the physiologic degradation rate from a few days up to about 20 years. Moreover, by introducing precious metals either within the alloy, or as a part of the metallic particles in combination with less precious metal particles, or alternatively by applying a currency for example with an appropriate electrode or similar device, the degradation of the metallic particles can substantially be altered. Using a metal also allows to utilize the mechanical strength of these compounds and to realize tailored implants that both address the mechanical requirements e.g. immediately after implantation for supportive functions, as well as the biodegradability for later provision or facilitation of tissue ingrowth and incorporation of the residual implant material, if any, into the bone or other tissue.

For example, the composition according to the exemplary embodiments of the present invention can rationally be tailored by suitably adjusting the metal composition to induce a controlled corrosion. Corrosion can occur when two metals, with different potentials, are in electrical contact while immersed or at least in contact in an electrically conducting corrosive liquid, like physiologic fluids. Because the metals have different natural potentials in the liquid, a current will flow from the anodic (more electronegative) metal to the cathodic (more electropositive) metal, which will increase the corrosion of the anode. This additional corrosion is also called bimetallic corrosion. It is also referred to as a galvanic corrosion, dissimilar metal corrosion or contact corrosion. In general, the degradation reactions which occur are similar to those that would occur on a single, uncoupled metal, but the rate of attack is increased, sometimes dramatically. With some exemplary metal combinations, the change in the electrode potential in the couple potential can induce corrosion which would not have occurred in the uncoupled state (e.g. pitting). The effect of coupling the two metals together can increase the corrosion rate of the anode and reduces or even suppresses corrosion of the cathode. Mostly, e.g., bimetallic corrosion can occur in solutions containing dissolved oxygen, and in most neutral and alkaline liquids the primary cathodic reaction is the reduction of dissolved oxygen, while in acidic liquids the cathodic reaction is often the reduction of hydrogen ions to hydrogen gas. Under an uncoupled corrosion, the anodic and cathodic reactions occur at small, local areas on the metal. In a bimetallic couple, the cathodic reaction is more, or totally, on the electropositive member of the couple and the anodic reaction is mostly, or totally, on the electronegative component of the couple.

Using these exemplary principles, the corrosion applied to the metallic particles in the cured compositions of the exemplary embodiments of the present invention can be a rationally tailored corrosion that can be verified by selecting suitable metallic particles and/or combinations thereof with regard to their electronegativity or electropositivity.

According to further exemplary embodiments of the present invention, the particles may have shapes such as tubes, fibers, fibrous materials or wires or spherical or dendritic or any regular or irregular particle form, and the preferred particle sizes are in, but not limited to, a range of about 1 nm (nanometer) up to 8000 μm (micrometer), preferably nano- or microsized particles.

The metallic material particles useful according to certain exemplary embodiments of the present invention can have an average (D50) particle size from about 0.5 nm to 5000 μm, preferably below about 1000 μm, such as from about 0.5 nm to 1,000 μm, or below 500 nm, such as from about 0.5 nm to 500 nm, or from about 500 nm to 400 nm. Preferred D50 particle size distributions can be in a range of about 10 nm up to 1000 μm, such as between about 25 nm and 600 μm or even between about 30 nm and 250 μm. Exemplary particle sizes and particle distribution of nano-sized particles may be determined by spectroscopic methods such as photo correlation spectroscopy, or by light scattering or laser diffraction techniques.

Concerning the exemplary corrosion control with regard to the metallic material particles, basically two approaches toward implant design may be used. The first exemplary approach can be the combination of first metal or metal alloy particles with identical or similar electronegativity together with at least one second entity of metal or metal alloy particles with a different electronegativity that is sufficient to affect the corrosion rate of the first particles. The second exemplary approach may be based on selecting particles that are alloyed, for example in nano-alloys, or core/shell particles or metal particles coated with a different metal that impacts the corrosion of one of its constituents. However, any combination of the foregoing approaches may also be used according to the present invention. For example, in one exemplary embodiment, magnesium particles can be combined with Ag or Au particles whereby the presence of a non-precious and precious metal would result in a rapidly corrodible or erodible combination. In another exemplary embodiment, magnesium particles coated with magnesium oxide can comprise a different corrosion rate compared to magnesium particles that are coated with silver oxide.

According to another exemplary embodiment of the present invention, the metallic material particles comprises a mixture of at least one first metallic material and at least one second metallic material, the first metallic material being more electronegative than the second metallic material, such that the first and second metallic material particles form a local cell at their contact surfaces. In such an embodiment, the less noble metal is preferentially degraded in-vivo.

In another exemplary embodiment of the present invention, the size and surface-to-volume ratio of the metallic particles may be used to control the corrosion rate. For example, when using the same degradable metal or metal alloy particle but in different sizes, the smaller particles or those with a higher surface-to-volume ratio are typically prone to a higher corrosion rate. Therefore, even using the same metallic basically still allows to tailor the corrosion rate by selecting the appropriate particle size or combination of particle sizes. However, also a combination of different material composition as well as different particles comprising significantly different surface-to-volume ratios can be combined. Preferably, the exemplary composition used comprises particles including metals with different electronegativities to tailor the basic corrosion rate of the implant with an appropriate alloy.

In exemplary embodiments, it can be preferable to have a rationally designed distribution of the metallic material particles and the curable, matrix-forming material within the final implant body. Such a distribution may e.g. be influenced by selecting appropriate amounts and sizes of the materials used.

The metallic particles as described herein can be combined in the compositions of the present invention with a curable matrix-forming, non-particulate material.

### Exemplary Curable Matrix-Forming, Non-Particulate Material

Exemplary Polymeric and Polymer-Solvent Systems

According to an exemplary embodiment, the exemplary composition as described herein can include an organic material as the curable matrix-forming, non-particulate material, preferably a polymer-solvent system. The exemplary embodiment of the polymer-solvent system can comprise a mixture of at least one polymer and at least one solvent or plasticizer.

For example, the exemplary organic material may comprise an oligomer, polymer or copolymer such as a poly(meth)acrylate, unsaturated polyester, saturated polyester, polyolefines, polyethylene, polypropylene, polybutylene, alkyd resins, epoxy-polymers or resins, polyamide, polyimide, polyetherimide, polyamideimide, polyesterimide, polyester amide imide, polyurethane, polycarboxylate, polycarbonate, polystyrene, polyphenol, polyvinyl ester, polysilicone, polyacetal, cellulosic acetate, polyvinylchloride, polyvinyl acetate, polyvinyl alcohol, polysulfone, polyphenylsulfone, polyethersulfone, polyketone, polyetherketone, polybenzimidazole, polybenzoxazole, polybenzothiazole, polyfluorocarbons, polyphenylene ether, polyarylate, or cyanatoester-polymers, and any of the copolymers and any mixtures thereof.

One exemplary option is to use a biocompatible, but non-degradable polymer, such as polymethylmethacrylate and/or other acrylic co-polymers, preferably acrylic-terminated butadiene-styrene block copolymers, or cyanoacrylates, polyetherketone or polyetheretherketone, pre-polymers or any mixture thereof, that is dissolved in a biocompatible solvent and can be hardened in-situ or ex-situ. Alternatively, a biodegradable polymer may be used.

According to another exemplary embodiment of the present invention, the organic material comprises a biocompatible and/or biodegradable polymer or copolymer such as collagen, albumin, gelatin, hyaluronic acid, starch, cellulose, methylcellulose, hydroxypropylcellulose, hydroxypropylmethyl-cellulose, carboxymethylcellulose-phthalate; gelatine, casein, dextrane, polysaccharide, fibrinogen, poly(D,L lactide), poly(D,L-lactide-co-glycolide), poly(glycolide-co-trimethylene carbonates), poly(glycolide), poly(hydroxybutylate), poly(alkylcarbonate), poly(a-hydroxyesters), poly(ether esters), poly(orthoester), polyester, poly(hydroxyvaleric acid), polydioxanone, poly(ethylene terephtalate), poly(maleic acid), poly(malic acid), poly(tartaric acid), polyanhydride, polyphosphazene, poly(amino acids), polypeptides, polycaprolactones, poly(propylene fumarates), poly(ester amides), poly(ethylene fumarates), poly(hydroxy butyrates), and polyurethanes, or mixtures thereof. In such exemplary embodiments, the organic material may be selected from partially or substantially completely biodegradable polymers.

Further polymers which may be used include, for example, poly(meth)acrylate, unsaturated polyester, saturated polyester, polyolefines such as polyethylene, polypropylene, polybutylene, alkyd resins, epoxy-polymers or resins, polyamide, polyimide, polyetherimide, polyamideimide, polyesterimide, polyester amide imide, polyurethane, polycarbonate, polystyrene, polyphenol, polyvinyl ester, polysilicone, polyacetal, cellulosic acetate, polyvinylchloride, polyvinyl acetate, polyvinyl alcohol, polysulfone, polyphenylsulfone, polyethersulfone, polyketone, polyetherketone, polybenzimidazole, polybenzoxazole, polybenzothiazole, polyfluorocarbons, polyphenylene ether, polyarylate, cyanatoester-polymers, and mixtures or copolymers of any of the foregoing.

In certain exemplary embodiments, the polymer material can be selected from poly(meth)acrylates based on mono(meth)acrylate, di(meth)acrylate, tri(meth)acrylate, tetra-acrylate and pentaacrylate monomers; as well as mixtures, copolymers and combinations of any of the foregoing.

In the exemplary embodiments of compositions comprising a polymer-solvent system, the solvent or plasticizer can be added in an amount sufficient to soften the polymer, but not liquefy the polymer. This can be sufficient to render the composition moldable for in-situ or ex-situ applications. Preferably, the exemplary composition can be in the form of a viscous paste with viscosities (at about 20° C.) in a range of about 200 to 800 Pa·s (Pascalseconds), more preferably in a range of about 400 to 600 Pa·s.

Alternatively, a sufficient quantity of liquid solvent can added to the polymer to liquefy and/or dissolve the polymer, rendering the composition sufficiently flowable for pouring into a bone defect such as a fissure in-vivo, or pouring into a mold ex-vivo. Preferably, such compositions may have a viscosity (at about 20° C.) in a range of about 200 Pa·s to about 400 Pa·s, more preferred of about 300 Pa·s to 500 Pa·s.

In addition, it may be preferred in polymer-solvent systems wherein the plasticizer is a solvent that it has solubility in an aqueous medium, ranging from miscible to dispersible. This can facilitate the hardening of the composition for example by extraction or diffusion of the solvent into (aqueous) body fluids in-vivo, e.g. blood, lymph, serum, or other tissue fluids.

In certain exemplary embodiments of the present invention, the solvent or plasticizer may be, for example, selected from at least one of water, an alcohol, acetone, ethyl lactate, ethyl acetate, ethyl formate, acetyltributylcitrate, triethyl citrate, tetrahydrofuran, toluene, and n-methyl-2-pyrrolidone (NMP), or other suitable solvents, such as ethyl acetoacetate or a mixture of ethyl acetoacetate and ethanol, or plasticizers. The solvent or plasticizer is preferably biocompatible, i.e. substantially non-toxic or at least exhibiting a very low toxicity. Typically, such polymer-solvent based compositions are curable or may be hardened by extraction of the solvent or plasticizer, such as diffusion of the solvent or plasticizer into an aqueous medium ex-vivo, or into body fluids in-vivo. Alternatively, the extraction may involve removal of the solvent or plasticizer by e.g. heat or pressure treatments such as drying, freeze-drying, or evaporation.

Monomer-Polymerization

According to still another exemplary embodiment of the present invention, the curable matrix-forming, non-particulate material in an exemplary embodiment can be an organic material comprising at least one polymerizable or crosslinkable monomer, for example a monofunctional monomer or a polyfunctional monomer.

According to certain exemplary embodiment, the monofunctional monomer may include at least one of methyl acrylate, methyl methacrylate, ethyl acrylate, ethyl methacrylate, butyl acrylate, butyl methacrylate, acryl acrylate, acryl methacrylate, hydroxyethyl acrylate, hydroxyethyl methacrylate, methoxyethyl acrylate, and methoxyethyl methacrylate.

The polyfunctional monomer may include at least one of bifunctional aliphatic acrylates, bifunctional aliphatic methacrylates, bifunctional aromatic acrylates, bifunctional aromatic methacrylates, trifunctional aliphatic acrylates, trifunctional aliphatic methacrylates, tetrafunctional acrylates, and tetrafunctional methacrylates, such as triethylene glycol diacrylate, triethylene glycol dimethacrylate, 2,2-bis(4-methacryloxyphenyl)propane, 2,2-bis(4-methacryloxyethoxyphenyl)propane, 2,2-bis(4-methacryloxypolyethoxyphenyl]propane, 2,2-bis[4-(3-methacryloxy-2-hydroxypropoxy)-phenyl]propane, di(methacryloxyethyl)trimethylhexamethylene diurethane, tetramethylolmethane tetraacrylate, and tetramethylolmethane tetramethacrylate, or a di(meth)acrylate, such as urethane dimethacrylate, ethyleneglycol dimethacrylate, (2,2-bis[4-(2-hydroxy-3-methacryloxypropoxy)phenyl]propane (BIS-GMA), (2,2-bis[4-(methacryloxy)phenyl]propane (BIS-MA), 2,2-bis(4-methacryloxyphenyl)propane, 1,2-butanediol-diacrylate, 1,4-butanediol-diacrylate, 1,4-butanediol-dimethacrylate, 1,4-cyclo-hexanediol-dimethacrylate, 1,10-decanediol-dimethacrylate, diethylene-glycol-diacrylate, dipropyleneglycol-diacrylate, dimethylpropanediol-dimethacrylate, triethyleneglycol-dimethacrylate (TEGDMA), tetraethyleneglycol-dimethacrylate, 1,6-hexanediol-diacrylate, 1,6-bis-[2-methacryloxyethoxycarbonylamino]-2,2,4-trimethylhexane (UDMA), neopentylglycol-diacrylate, polyethyleneglycol-dimethacrylate, tripropyleneglycol-diacrylate, 2,2-bis-[4-(2-acryloxyethoxy)phenyl]-propane, 2,2-bis-[4-(2-hydroxy-3-methacryloxypropoxy)phenyl]propane, bis(2-methacryloxyethyl)N,N-1,9-nonylene-biscarbamate, 1,4-cyclohexanedimethanol-dimethacrylate, and diacrylic urethane oligomers. Also, mixtures of monofunctional and polyfunctional monomers may be used.

For curing or hardening the composition of such exemplary embodiments, the composition can additionally comprise a polymerization catalyst, an initiator, or an accelerator. For example, the composition may comprise a catalyst such as a photoinitiator e.g. camphorquinone, an accelerator such as ethyl-p-dimethylaminobenzoate (DMAB) or N,N-dimethylaminoethyl methacrylate (DMAEMA), or a redox catalyst, preferably selected from a combination of an amine and a peroxide, a combination of a sulfinic acid and a peroxide, or a combination of an other material and a peroxide.

In exemplary embodiments, the peroxide includes at least one of a diacyl peroxide such as benzoyl peroxide, p-chlorobenzoyl peroxide, 2,4-dichlorobenzoyl peroxide, acetyl peroxide, and lauroyl peroxide; or a hydroperoxide such as t-butyl hydroperoxide, cumene hydroperoxide, and 2,5-dimethylhexane 2,5-dihydroperoxide; or a ketone peroxide such as methyl ethyl ketone peroxide; or a peroxycarbonate such as t-butyl peroxybenzoate.

Furthermore, the amine can include at least one of N,N-bis-(2-hydroxyethyl)4-methylaniline, N,N-bis-(2-hydroxyethyl)-3,4-dimethylaniline, N,N-bis-(2-hydroxyethyl)-3,5-dimethylaniline, N-methyl-N-(2-hydroxyethyl)-4-methylaniline, 4-methylaniline, N,N-dimethyl-p-toluidine (DMPT), N,N-dimethylaniline, and triethanolamine.

In still further exemplary embodiments where sulfinic acid redox catalysts are used, the sulfinic acid includes at least one of p-toluenesulfinic acid, benzenesulfinic acid, and salts thereof. Furthermore, an other material can be combined with the peroxide, such as cobalt naphthenate, cobalt octanate, trimethyl barbituric acid, and a trialkyl boron.

According to yet another exemplary embodiment of the present invention, the composition can be a two component system, whereas the composition may be divided into two parts, the amine or sulfinic acid is incorporated into one part whereas the peroxide is incorporated into the other part, and the both parts are to be mixed at the time of use.

In further exemplary embodiments, the curable matrix-forming material may also be selected from commercially available biomedical cement materials such as, for example, Palacos®, Palamed®, Osteopal®, or Copal® cements available from Heraeus Medical GmbH, Germany, or Simplex® P of Howmedica, and mixed with degradable metallic particulate materials as described herein. Such matrix materials and similar ones based on the organic materials described herein can for example be prepared by mixing and crosslinking solid polymer with liquid monomers, particularly polymethylacrylat and polymethylmethacrylat with methylmethacrylat, or methylmethacrylate-styrene copolymers mixed with polymethylmethacrylate (PMMA).

In certain exemplary embodiments of the present invention, curable matrix materials may be based on polypropylene fumarate, or polymer systems known from dental applications, such as Bis-GMA based acrylic materials, for example modified Bis-GMA acrylic acids, as well as Poly(vinyl phosphonic acid), copolymers of the acrylic, itaconic, maleic and phosphonic vinyl acids based materials, or poly-fluorinated acrylic monomers and oligomers.

Sol-Gel-Systems

According to a further exemplary embodiment of the present invention, the curable matrix-forming, non-particulate material of an exemplary embodiment can include precursor compounds of an inorganic-organic hybrid material, processible by sol-gel processing. The sol-gel-processing can be either a hydrolytic or non-hydrolytic sol-gel processing.

The precursor compounds processible by sol-gel processing include at least one metal alkoxide. For example, the metal alkoxide can be selected from at least one of silicon alkoxides, tetraalkoxysilanes, oligomeric forms of tetraalkoxysilanes, alkylalkoxysilanes, aryltrialkoxysilanes, (meth)acrylsilanes, phenylsilanes, oligomeric silanes, polymeric silanes, epoxysilanes; fluoroalkylsilanes, fluoroalkyltrimethoxysilanes, or fluoroalkyltriethoxysilanes.

In such exemplary embodiments, the composition may further comprise, at least one crosslinking agent including at least one of isocyanates, silanes, (meth)acrylates, 2-hydroxyethyl methacrylate, propyltrimethoxysilane, 3-(trimethylsilyl)propyl methacrylate, isophoron diisocyanate, HMDI, diethylenetriaminoisocyanate, 1,6-diisocyanatohexane, or glycerin.

In another exemplary embodiment, the metal alkoxide includes a hydrolytically condensable, organically modified trialkoxysilane which contains free-radically polymerizable acrylate or methacrylate groups or cyclic groups capable of ring opening polymerization. Suitable materials include, e.g. those based on polysilicid acid modified with polymerizable alkoxy groups or cyclic siloxanes and a mixture of Bis-GMA and 2-hydroxyethyl methacrylate (HEMA). These materials can be cured by hydrolysis and condensation with simultaneous radical polymerization of the resultant alcohols. Functionalized trialkoxysilanes of the R—Si(OR′)₃ type may also be used, which can condensate, resulting in polysilsesquioxanes RSiO_{3/2}, or which can be co-condensated with other alkoxysilanes or metal alkoxides.

Methacrylates may also be used in combination with e.g. tetraethylorthosilicate (TEOS) to provide PMMA-silica hybrides after curing by polymerization and co-condensation.

An overview on several of these precursors for inorganic-organic hybrid materials suitable for the compositions of the exemplary embodiment of the present invention is described in N. Moszner and S. Klapdohr, Nanotechnology for dental composites, Int. J. of Nanotechnology, vol. 1, No. ½, 2004, 130-156, and the references cited therein. All materials referred to and mentioned therein are in principle also suitable for use as the matrix-forming material in the compositions of the present invention. For example, hydrolysable and condensable trialkoxysilanes bearing methacrylate groups can be used, which are connected to the Si-atom via spacers, and silanediacrylates can be preferred materials which can be hydrolysed and condensated into fluid sols, and cured by e.g. visible light by polymerization of the methacrylate functions.

The precursor compounds of an inorganic-organic hybrid material processible by sol-gel processing may be conventional sol/gel-forming components. The sol/gel-forming components are typically provided in the form of a sol which may comprise a solvent, and which can be cured or hardened by condensation into a gel such as an aerogel or xerogel.

In these exemplary embodiments degradable and non degradable metallic particles selected as described above can be combined and mixed with the sol/gel-forming components, or specifically only degradable or non-degradable particles can be used. Optionally, the gel obtained after curing is dissolvable in physiologic fluids, or porous.

In certain exemplary embodiments, the sol/gel forming components can include metal oxides, metal carbides, metal nitrides, metaloxynitrides, metalcarbonitrides, metaloxycarbides, metaloxynitrides, and metaloxycarbonitrides of the above mentioned metals, or any combinations thereof. These compounds, preferably as colloidal particles, can be reacted with oxygen-containing compounds, e.g. alkoxides to form a sol/gel.

In exemplary embodiments of the third aspect of the present invention, the sols are derived from at least one sol/gel forming component selected from alkoxides, metal alkoxides, colloidal particles, particularly metal oxides and the like. The metal alkoxides useful as sol/gel forming components in this invention, are well-known chemical compounds that are used in a variety of applications. They can for example have the general formula M(OR)ₓ, wherein M is any metal from a metal alkoxide which e.g. will hydrolyze and polymerize in the presence of water. R is an alkyl radical of 1 to 30 carbon atoms, which may be straight chained or branched, and x has a value equivalent to the metal ion valence. Preferred in certain exemplary embodiments of the present invention, are such metal alkoxides as Si(OR)₄, Ti(OR)₄, Al(OR)₃, Zr(OR)₃ and Sn(OR)₄. Specifically, R can be the methyl, ethyl, propyl or butyl radical. Further examples of suitable metal alkoxides are Ti(isopropoxy)₄, Al(isopropoxy)₃, Al(sec-butoxy)₃, Zr(n-butoxy)₄ and Zr(n-propoxy)₄.

Silicon alkoxides such as tetraalkoxysilanes may be used in exemplary embodiments, wherein the alkoxy may be branched or straight chained and may contain 1 to 25 carbon atoms, e.g. tetramethoxysilane (TMOS), tetraethoxysilane (TEOS) or tetra-n-propoxysilane, as well as oligomeric forms thereof. Also suitable are alkylalkoxysilanes, wherein alkoxy is defined as above and alkyl may be a substituted or unsubstituted, branched or straight chain alkyl having 1 to 25 carbon atoms, e.g. methyltrimethoxysilane (MTMOS), methyltriethoxysilane, ethyltriethoxysilane, ethyltrimethoxysilane, methyltripropoxysilane, methyltributoxysilane, propyltrimethoxysilane, propyltriethoxysilane, isobutyltriethoxysilane, isobutyltrimethoxy silane, octyltriethoxysilane, octyltrimethoxysilane, commercially available from Degussa AG, Germany, methacryloxydecyltrimethoxysilane (MDTMS); aryltrialkoxysilanes such as phenyltrimethoxysilane (PTMOS), phenyltriethoxysilane, commercially available from Degussa AG, Germany; phenyltripropoxysilane, and phenyltributoxysilane, phenyl-tri-(3-glycidyloxy)-silane-oxide (TGPSO), 3 aminopropyltrimethoxysilane, 3-aminopropyl-triethoxysilane, 2 aminoethyl 3 aminopropyltrimethoxysilane, triaminofunctional propyltrimethoxysilane (Dynasylan® TRIAMO, available from Degussa AG, Germany), N-(n-butyl)-3-aminopropyltrimethoxysilane, 3 aminopropylmethyl-diethoxysilane, 3-glycidyloxypropyltrimethoxysilane, 3 glycidyloxypropyltriethoxy-silane, vinyltrimethoxysilane, vinyltriethoxysilane, 3 mercaptopropyltrimethoxy-silane, bisphenol-A-glycidylsilanes; (meth)acrylsilanes, phenylsilanes, oligomeric or polymeric silanes, epoxysilanes; fluoroalkylsilanes such as fluoroalkyltrimethoxysilanes, fluoroalkyltriethoxysilanes with a partially or fully fluorinated, straight chain or branched fluoroalkyl residue of 1 to 20 carbon atoms, e.g. tridecafluoro-1,1,2,2 tetrahydrooctyltriethoxysilane and modified reactive fluoroalkylsiloxanes available from Degussa AG under the trademarks Dynasylan® F8800 and F8815; as well as any mixtures of the foregoing.

The sol/gel components may also be selected from colloidal metal oxides, preferably those colloidal metal oxides which are stable long enough to be able to combine them with the other sol/gel components. Such colloidal metal oxides can include SiO₂, Al₂O₃, MgO, ZrO₂, TiO₂, SnO₂, ZrSiO₄, ZrO(NO₃)₂, B₂O₃, La₂O₃ and Sb₂O₅. Further examples for the sol/gel forming component are aluminiumhydroxide sols or gels, aluminiumtri-sec-butylat, AlOOH-gels and the like.

Some of these exemplary colloidal sols are acidic in the sol form and, therefore, when used in conjunction with this invention during curing by e.g. hydrolysis, additional acid need not be added to the hydrolysis medium. These colloidal sols can also be prepared by a variety of methods. For example, titania sols having a particle size in the range of about 5 to 150 nm can be prepared by the acidic hydrolysis of titanium tetrachloride, by peptizing hydrous TiO₂ with tartaric acid and, by peptizing ammonia washed Ti(SO₄)₂ with hydrochloric acid. See Weiser, Inorganic Colloidal Chemistry, Vol. 2, p. 281 (1935). For the purposes of this invention, and in order to preclude the incorporation of contaminants in the sols, it is preferred to hydrolyze the alkyl orthoesters of the metals in an acid pH range of 1 to 3, in the presence of a water miscible solvent, wherein the colloid is present in the dispersion in an amount of 0.1 to 10 weight percent.

In case the sol is formed by a hydrolytic sol/gel-process, the molar ratio of the added water and the sol/gel forming components such as alkoxides, oxides, acetates, nitrides or combinations thereof, is preferably in the range of 0.001 to 100, preferably from 0.1 to 80, more preferrably from about 0.2 to 30.

In an exemplary hydrolytic sol/gel processing procedure, the composition comprises a mixture of the sol/gel components and the metallic material particles in the presence of water, and optionally further solvents or mixtures thereof, and further additives may be added, such as surfactants, fillers and the like, as described in more detail hereinafter. Further additives like crosslinkers may be added to the composition, as well as catalysts for controlling the hydrolysis rate of the sol or for controlling the crosslinking rate. Such catalysts are also described in further detail hereinbelow. Such processing is similar to sol/gel processing conventionally known.

Non-hydrolytic sols are similarly made as described herein, however essentially in the absence of water. In nonhydrolytic sol/gel processes, the use of metal alkoxides and carboxylic acids and their derivatives or carboxylic acid functionalized metallic material particles may also be used. Suitable carboxylic acids are acetic acid, acetoacetic acid, formic acid, maleic acid, crotonic acid, succinic acid, acrylic acid, methacrylic acid, partially or fully fluorinated carboxylic acids, their anhydrides and esters, e.g. methyl- or ethylesters, and any mixtures of the foregoing. In the case of acid anhydrides, it is often preferred to use these anhydrides in admixture with anhydrous alcohols, wherein the molar ratio of these components determines the amount of residual acetoxy groups at the silicon atom of the alkylsilane employed. Non-hydrolytic sol/gel processing in the absence of water may be accomplished by reacting alkylsilanes or metal alkoxides with anhydrous organic acids, acid anhydrides or acid esters, or the like.

Typically, according to the degree of cross-linking desired during curing, either acidic or basic catalysts are applied, particularly in hydrolytic sol/gel processes. Suitable inorganic acids are, for example, hydrochloric acid, sulfuric acid, phosphoric acid, nitric acid as well as diluted hydrofluoric acid. Suitable bases are, for example, sodium hydroxide, ammonia and carbonate as well as organic amines. Suitable catalysts in non-hydrolytic sol/gel processes are anhydrous halide compounds, for example BCl₃, NH₃, AlCl₃, TiCl₃ or mixtures thereof.

To effect the hydrolysis in hydrolytic sol/gel processing steps, the addition of solvents other than water may be used. Examples can be water-miscible solvents. It may be preferred to use water-miscible alcohols herein or mixtures of water-miscible alcohols. Especially suitable are alcohols such as methanol, ethanol, n propanol, isopropanol, n-butanol, isobutanol, t-butanol and lower molecular weight ether alcohols such as ethylene glycol monomethyl ether. It can also be preferable to use small amounts of non-water-miscible solvents such as toluene.

Further, in these exemplary embodiments, a cross-linker is added, the crosslinking agent being selected from, for example, isocyanates, silanes, diols, di-carboxylic acids, (meth)acrylates, for example such as 2-hydroxyethyl methacrylate, propyltrimethoxysilane, 3-(trimethylsilyl)propyl methacrylate, isophoron diisocyanate, polyols, glycerin and the like. Particularly preferred are biocompatible crosslinkers such as glycerin, diethylentriaminoisocyanate and 1,6-diisocyanatohexane.

In certain exemplary embodiment, the curable matrix-forming material may include a combination of any of the above described embodiments of the first, second and third aspects of the present invention. For example, hydrolytically condensable metal alkoxides used in sol-gel processing may include at least one polymerizable monofunctional or polyfunctional organic residue, which may be additionally or subsequently subjected to polymerization upon curing the composition, or polymerizable monomer materials may be combined with polymer-solvent systems.

In the exemplary embodiments of the present invention, the composition can be cured by drying, solvent extraction, radiation, such as visible light, UV or IR radiation, heat, polymerization or chemical crosslinking.

In addition, the compositions of particular exemplary embodiments may further comprise conventional additives such as a crosslinker, a silane coupling agent, a plasticizer, a solvent, a filler, preferably an inorganic filler such as silica powder, quartz, glass beads, aluminum oxide, ceramics, salts, hydroxyl apatite; a stabilizer such as hydroquinone, hydroquinone monomethyl ether, t-butyl paracresol and hydroxy methoxybenzophenone, a pigment, or a beneficial agent as further described herein below, which may optionally be configured to be released in-vivo from the final implant

According to a further exemplary embodiment of the present invention, the particles of metallic material comprise at least about 5 wt.-%, preferably from about 1 to 99 wt.-%, more preferred 10 to 80 wt.-%, most preferred 40 to 75 wt-% of the composition. Furthermore, the metallic material particles can be modified with a coupling agent, preferably a silane coupling agent such as vinyl trichlorosilane, vinyl triethoxysilane, vinyl trimethoxysilane, vinyl tris(beta-methoxyethoxy)silane, and gamma-methacryloxypropyl trimethoxysilane.

According to another exemplary embodiment of the present invention, a method of filling a cavity in a living organism is provided, which in an exemplary embodiment comprises the filling of the cavity with the implant composition as described herein in-vivo, and subsequently curing the composition.

According to an alternative exemplary embodiment of the present invention, the method comprises shaping the composition as described herein ex-vivo into a desired shape for filling the cavity, for example in a mold, curing the composition; and subsequently implanting the cured composition into the cavity in the living organism. For example, the cavity may be a defect or wound in a bone, or tooth or cartilage of a living organism.

According to a particular exemplary embodiment of the present invention, the cured composition after implantation facilitates and enables the formation and organization of tissue, preferably osteoinduction, osteoconduction and formation of natural bone minerals “guided” by the implant fine-structure.

If the composition of the present invention is to be shaped and cured ex-situ before implantation, this may be done by any suitable conventional method. Appropriate techniques include molding the composition in a mold or replica form of the defect to be filled with the desired design. In addition, for example an injection molding processes can be applied. Other exemplary methods include compression molding, compacting, dry pressing, cold isostatic pressing, hot pressing, uniaxial or biaxial pressing, extrusion molding, gel casting, slip casting and tape casting and the like.

Functionalization

According to an exemplary embodiment of the present invention, additional functions may be provided in the composition or the cured implant or filling by incorporating beneficial agents into the composition before or after curing, as desired. Beneficial agents can be selected from biologically active agents, pharmacological active agents, therapeutically active agents, diagnostic agents or absorptive agents or any mixture thereof. Furthermore, if shaped ex-situ, the implant may optionally be coated with beneficial agents partially or completely.

Biologically, therapeutically or pharmaceutically active agents according to the present invention may include a drug, pro-drug or even a targeting group or a drug comprising a targeting group. The active agents may be in crystalline, polymorphous or amorphous form or any combination thereof in order to be used in the present invention. Suitable therapeutically active agents may be selected from the group of enzyme inhibitors, hormones, cytokines, growth factors, receptor ligands, antibodies, antigens, ion binding agents such as crown ethers and chelating compounds, substantial complementary nucleic acids, nucleic acid binding proteins including transcriptions factors, toxines and the like.

Examples of active agents can be, for example, cytokines such as erythropoietine (EPO), thrombopoietine (TPO), interleukines (including IL-1 to IL-17), insulin, insulin-like growth factors (including IGF-1 and IGF-2), epidermal growth factor (EGF), transforming growth factors (including TGF-alpha and TGF-beta), human growth hormone, transferrine, low density lipoproteins, high density lipoproteins, leptine, VEGF, PDGF, ciliary neurotrophic factor, prolactine, adrenocorticotropic hormone (ACTH), calcitonin, human chorionic gonadotropin, cortisol, estradiol, follicle stimulating hormone (FSH), thyroid-stimulating hormone (TSH), leutinizing hormone (LH), progesterone, testosterone, toxines including ricine and further active agents such as those included in Physician's Desk Reference, 58th Edition, Medical Economics Data Production Company, Montvale, N.J., 2004 and the Merck Index, 13th Edition (particularly pages Ther-1 to Ther-29), all of which are incorporated herein by reference.

In a exemplary embodiment, the therapeutically active agent is selected from the group of drugs for the therapy of oncological diseases and cellular or tissue alterations. Suitable therapeutic agents are, e.g., antineoplastic agents, including alkylating agents such as alkyl sulfonates, e.g., busulfan, improsulfan, piposulfane, aziridines such as benzodepa, carboquone, meturedepa, uredepa; ethyleneimine and methylmelamines such as altretamine, triethylene melamine, triethylene phosphoramide, triethylene thiophosphoramide, trimethylolmelamine; so-called nitrogen mustards such as chlorambucil, chlornaphazine, cyclophosphamide, estramustine, ifosfamide, mechlorethamine, mechlorethaminoxide hydrochloride, melphalan, novembichin, phenesterine, prednimustine, trofosfamide, uracil mustard; nitroso urea-compounds such as carmustine, chlorozotocin, fotenmustine, lomustine, nimustine, ranimustine; dacarbazine, mannomustine, mitobranitol, mitolactol; pipobroman; doxorubicin and cis-platinum and its derivatives, and the like, combinations and/or derivatives of any of the foregoing.

In a further exemplary embodiment, the therapeutically active agent is selected from the group of anti-viral and anti-bacterial agents such as aclacinomycin, actinomycin, anthramycin, azaserine, bleomycin, cuctinomycin, carubicin, carzinophilin, chromomycines, ductinomycin, daunorubicin, 6-diazo-5-oxn-1-norieucin, doxorubicin, epirubicin, mitomycins, mycophenolsäure, mogalumycin, olivomycin, peplomycin, plicamycin, porfiromycin, puromycin, streptonigrin, streptozocin, tubercidin, ubenimex, zinostatin, zorubicin, aminoglycosides or polyenes or macrolid-antibiotics, and the like, combinations and/or derivatives of any of the foregoing.

In a further exemplary embodiment, the therapeutically active agent can be selected from the group of radio-sensitizer drugs. In a further exemplary embodiment, the therapeutically active agent can be selected from the group of steroidal or non-steroidal anti-inflammatory drugs. In yet further exemplary embodiment, the therapeutically active agent can be selected from agents referring to angiogenesis, such as e.g. endostatin, angiostatin, interferones, platelet factor 4 (PF4), thrombospondin, transforming growth factor beta, tissue inhibitors of the metalloproteinases-1, -2 and -3 (TIMP-1, -2 and -3), TNP-470, marimastat, neovastat, BMS-275291, COL-3, AG3340, thalidomide, squalamine, combrestastatin, SU5416, SU6668, IFN-[alpha], EMD121974, CAI, IL-12 and IM862 and the like, combinations and/or derivatives of any of the foregoing.

In still further exemplary embodiment, the therapeutically-active agent can be selected from the group of nucleic acids, wherein the term nucleic acids also comprises oligonucleotides wherein at least two nucleotides are covalently linked to each other, for example in order to provide gene therapeutic or antisense effects. Nucleic acids can preferably comprise phosphodiester bonds, which also comprise those which are analogues having different backbones. Analogues may also contain backbones such as, for example, phosphoramide (Beaucage et al., Tetrahedron 49(10):1925 (1993) and the references cited therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)); phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidit-compounds (see Eckstein, Oligonucleotides and Analogues: A Practical Approach, Oxford University Press), and peptide-nucleic acid-backbones and their compounds (see Egholm, J. Am. Chem. Soc. 114:1895 (1992); Meier et al., Chem. Int. Ed. Engl: 31:1008 (1992); Nielsen, Nature, 365:566 (1993); Carlsson et al., Nature 380:207 (1996), wherein these references are incorporated by reference herein. further analogues are those having ionic backbones, see Denpcy et al., Proc. Natl. Acad. Sci. USA 92:6097 (1995), or non-ionic backbones, see U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996), and non-ribose-backbones, including those which are described in U.S. Pat. Nos. 5,235,033 and 5,034,506, and in chapters 6 and 7 of ASC Symposium Series 580, “Carbohydrate Modifications in Antisense Research”, Ed. Y. S. Sanghui and P. Dan Cook. The nucleic acids having one or more carbocylic sugars are also suitable as nucleic acids for use in the present invention, see Jenkins et al., Chemical Society Review (1995), pages 169 to 176 as well as others which are described in Rawls, C & E News, 2 Jun. 1997, page 36, herewith incorporated by reference. Besides the selection of the nucleic acids and nucleic acid analogues known in the conventional, also any mixtures of naturally occurring nucleic acids and nucleic acid analogues or mixtures of nucleic acid analogues may be used.

In a further embodiment, the therapeutically active agent is selected from the group of metal ion complexes, as described in International Application Nos. PCT/US95/16377, PCT/US95/16377, PCT/US96/19900 and PCT/US96/15527 which are incorporated by reference herein in their entireties, whereas such agents reduce or inactivate the bioactivity of their target molecules, preferably proteins such as enzymes.

Exemplary therapeutically active agents are also anti-migratory, anti-proliferative or immune-supressive, anti-inflammatory or re-endotheliating agents such as, e.g., everolimus, tacrolimus, sirolimus, mycofenolate-mofetil, rapamycin, paclitaxel, actinomycine D, angiopeptin, batimastate, estradiol, VEGF, statines and others, their derivatives and analogues.

Further preferred are active agents or combinations of active agents selected from heparin, synthetic heparin analogs (e.g., fondaparinux), hirudin, antithrombin III, drotrecogin alpha; fibrinolytics such as alteplase, plasmin, lysokinases, factor XIIa, prourokinase, urokinase, anistreplase, streptokinase; platelet aggregation inhibitors such as acetylsalicylic acid [aspirin], ticlopidine, clopidogrel, abciximab, dextrans; corticosteroids; so-called non-steroidal anti-inflammatory drugs (NSAIDs); cytostatics such as alkaloides and podophyllum toxins such as vinblastine, vincristine; alkylating agents such as nitrosoureas, nitrogen lost analogs; cytotoxic antibiotics such as daunorubicin, doxorubicin and other anthracyclines and related substances, bleomycin, mitomycin; antimetabolites such as folic acid analogs, purine analogs or pyrimidine analogs; paclitaxel, docetaxel, sirolimus; platinum compounds such as carboplatin, cisplatin or oxaliplatin; amsacrin, irinotecan, imatinib, topotecan, interferon-alpha 2a, interferon-alpha 2b, hydroxycarbamide, miltefosine, pentostatin, porfimer, aldesleukin, bexaroten, tretinoin; antiandrogens and antiestrogens; agents for stimulating angiogenesis in the myocardium such as vascular endothelial growth factor (VEGF), basic fibroblast growth factor (bFGF), non-viral DNA, viral DNA, endothelial growth factors: FGF-1, FGF-2, VEGF, TGF; antibiotics, monoclonal antibodies, anticalins; stem cells, endothelial progenitor cells (EPC); digitalis glycosides, such as acetyl digoxin/metildigoxin, digitoxin, digoxin; cardiac glycosides such as ouabain, proscillaridin; antihypertensives such as CNS active antiadrenergic substances, e.g., methyldopa, imidazoline receptor agonists; calcium channel blockers of the dihydropyridine type such as nifedipine, nitrendipine; ACE inhibitors: quinaprilate, cilazapril, moexipril, trandolapril, spirapril, imidapril, trandolapril; angiotensin II antagonists: candesartancilexetil, valsartan, telmisartan, olmesartanmedoxomil, eprosartan; peripherally active alpha-receptor blockers such as prazosin, urapidil, doxazosin, bunazosin, terazosin, indoramin; vasodilatators such as dihydralazine, diisopropylamine dichloracetate, minoxidil, nitroprusside sodium; other antihypertensives such as indapamide, co-dergocrine mesylate, dihydroergotoxin methanessulfonate, cicletanin, bosentan, fludrocortisone; phosphodiesterase inhibitors such as milrinon, enoximon and antihypotensives such as in particular adrenergic and dopaminergic substances such as dobutamine, epinephrine, etilefrine, norfenefrine, norepinephrine, oxilofrine, dopamine, midodrine, pholedrine, ameziniummetil; and partial adrenoceptor agonists such as dihydroergotamine; fibronectin, polylysine, ethylene vinyl acetate, inflammatory cytokines such as: TGF, PDGF, VEGF, bFGF, TNF, NGF, GM-CSF, IGF-a, IL-1, IL 8, IL-6, growth hormone; as well as adhesive substances such as cyanoacrylates, beryllium, silica; and growth factors such as erythropoetin, hormones such as corticotropins, gonadotropins, somatropins, thyrotrophins, desmopressin, terlipressin, pxytocin, cetrorelix, corticorelin, leuprorelin, triptorelin, gonadorelin, ganirelix, buserelin, nafarelin, goserelin, as well as regulatory peptides such as somatostatin, octreotid; bone and cartilage stimulating peptides, bone morphogenetic proteins (BMPs), in particulary recombinant BMPs, such as recombinant human BMP-2 (rhBMP-2), bisphosphonate (e.g., risedronate, pamidronate, ibandronate, zoledronic acid, clodronsaure, etidronsaure, alendronic acid, tiludronic acid), fluorides such as disodium fluorophosphate, sodium fluoride; calcitonin, dihydrotachystyrol; growth factors and cytokines such as epidermal growth factor (EGF), platelet-derived growth factor (PDGF), fibroblast growth factors (FGFs), transforming growth factors-b (TGFs-b), transforming growth factor-a (TGF-a), erythropoietin (EPO), insulin-like growth factor-I (IGF-I), insulin-like growth factor-II (IGF-II), interleukin-1 (IL-1), interleukin-2 (IL-2), interleukin-6 (IL-6), interleukin-8 (IL-8), tumor necrosis factor-a (TNF-a), tumor necrosis factor-b (TNF-b), interferon-g (INF-g), colony stimulating factors (CSFs); monocyte chemotactic protein, fibroblast stimulating factor 1, histamine, fibrin or fibrinogen, endothelin-1, angiotensin II, collagens, bromocriptine, methysergide, methotrexate, carbon tetrachloride, thioacetamide and ethanol; as well as silver (ions), titanium dioxide, antibiotics and anti-infective drugs such as in particular β-lactam antibiotics, e.g., β-lactamase-sensitive penicillins such as benzyl penicillins (penicillin G), phenoxymethylpenicillin (penicillin V); β-lactamase-resistant penicillins such as aminopenicillins, e.g., amoxicillin, ampicillin, bacampicillin; acylaminopenicillins such as mezlocillin, piperacillin; carboxypenicillins, cephalosporins such as cefazoline, cefuroxim, cefoxitin, cefotiam, cefaclor, cefadroxil, cefalexin, loracarbef, cefixim, cefuroximaxetil, ceftibuten, cefpodoximproxetil, cefpodoximproxetil; aztreonam, ertapenem, meropenem; β-lactamase inhibitors such as sulbactam, sultamicillintosylate; tetracyclines such as doxycycline, minocycline, tetracycline, chlorotetracycline, oxytetracycline; aminoglycosides such as gentamicin, neomycin, streptomycin, tobramycin, amikacin, netilmicin, paromomycin, framycetin, spectinomycin; macrolide antibiotics such as azithromycin, clarithromycin, erythromycin, roxithromycin, spiramycin, josamycin; lincosamides such as clindamycin, lincomycin; gyrase inhibitors such as fluoroquinolones, e.g., ciprofloxacin, ofloxacin, moxifloxacin, norfloxacin, gatifloxacin, enoxacin, fleroxacin, levofloxacin; quinolones such as pipemidic acid; sulfonamides, trimethoprim, sulfadiazine, sulfalene; glycopeptide antibiotics such as vancomycin, teicoplanin; polypeptide antibiotics such as polymyxins, e.g., colistin, polymyxin-b, nitroimidazole derivates, e.g., metronidazole, tinidazole; aminoquinolones such as chloroquin, mefloquin, hydroxychloroquin; biguanids such as proguanil; quinine alkaloids and diaminopyrimidines such as pyrimethamine; amphenicols such as chloramphenicol; rifabutin, dapson, fusidic acid, fosfomycin, nifuratel, telithromycin, fusafungin, fosfomycin, pentamidine diisethionate, rifampicin, taurolidin, atovaquon, linezolid; virus static such as aciclovir, ganciclovir, famciclovir, foscarnet, inosine-(dimepranol-4-acetamidobenzoate), valganciclovir, valaciclovir, cidofovir, brivudin; antiretroviral active ingredients (nucleoside analog reverse-transcriptase inhibitors and derivatives) such as lamivudine, zalcitabine, didanosine, zidovudin, tenofovir, stavudin, abacavir; non-nucleoside analog reverse-transcriptase inhibitors: amprenavir, indinavir, saquinavir, lopinavir, ritonavir, nelfinavir; amantadine, ribavirine, zanamivir, oseltamivir or lamivudine, as well as any combinations and mixtures thereof.

In another exemplary embodiment of the present invention, the active agents are encapsulated in polymers, vesicles, liposomes or micelles.

Suitable diagnostically active agents for use with the exemplary embodiments of the present invention can be e.g. signal generating agents or materials, which may be used as markers. Such signal generating agents include materials which in physical, chemical and/or biological measurement and verification methods lead to detectable signals, for example in image-producing methods. It is not important for exemplary embodiments of the present invention whether the signal processing is carried out exclusively for diagnostic or therapeutic purposes. Exemplary imaging methods are for example radiographic methods, which are based on ionizing radiation, for example conventional X-ray methods and X-ray based split image methods such as computer tomography, neutron transmission tomography, radiofrequency magnetization such as magnetic resonance tomography, further by radionuclide-based methods such as scintigraphy, Single Photon Emission Computed Tomography (SPECT), Positron Emission Computed Tomography (PET), ultrasound-based methods or fluoroscopic methods or luminescence or fluorescence based methods such as Intravasal Fluorescence Spectroscopy, Raman spectroscopy, Fluorescence Emission Spectroscopy, Electrical Impedance Spectroscopy, colorimetry, optical coherence tomography, etc, further Electron Spin Resonance (ESR), Radio Frequency (RF) and Microwave Laser and similar methods.

Signal generating agents can be metal-based from the group of metals, metal oxides, metal carbides, metal nitrides, metal oxynitrides, metal carbonitrides, metal oxycarbides, metal oxynitrides, metal oxycarbonitrides, metal hydrides, metal alkoxides, metal halides, inorganic or organic metal salts, metal polymers, metallocenes, and other organometallic compounds, chosen from powders, solutions, dispersions, suspensions, emulsions. Exemplary metal based agents are especially nanomorphous nanoparticles from metals, metal oxides or mixtures there from. The metals or metal oxides used can also be magnetic; examples are—without excluding other metals—iron, cobalt, nickel, manganese or mixtures thereof, for example iron-platinum mixtures, or as an example for magnetic metal oxides, iron oxide and ferrites.

It is possible to use semi conducting nanoparticles, examples for this are semiconductors from group II-VI, group III-V, group IV. Group II-VI-semiconductors are for example MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, HgTe or mixtures thereof. Examples of group III-V semiconductors are for example GaAs, GaN, GaP, GaSb, InGaAs, InP, InN, InSb, InAs, AlAs, AlP, AlSb, AlS, and mixtures thereof are preferred. Germanium, lead and silicon are selected as exemplary of group IV semiconductors. The semiconductors can moreover also contain mixtures of semiconductors from more than one group, all groups mentioned above are included.

It is possible to select complex formed metal-based nanoparticles. Included here are so-called Core-Shell configurations, as described explicitly by Peng et al., “Epitaxial Growth of Highly Luminescent CdSe/CdS Core/Shell Nanoparticles with Photo stability and Electronic Accessibility”, Journal of the American Chemical Society, (1997) 119:7019-7029, and included herewith explicitly per reference. Preferred here are semi conducting nanoparticles, which form a core with a diameter of 1-30 nm, especially preferred of 1-15 nm, onto which other semi conducting nanoparticles crystallize in 1-50 monolayers, especially preferred are 1-15 monolayers. In this case core and shell can be present in any desired combinations as described above, in special embodiments CdSe and CdTe are preferred as the core and CdS and ZnS as the shell.

Further, exemplary signal producing metal-based agents can be selected from salts or metal ions, which preferably have paramagnetic properties, for example lead (II), bismuth (II), bismuth (III), chromium (III), manganese (II), manganese (III), iron (II), iron (III), cobalt (II), nickel (II), copper (II), praseodymium (III), neodymium (III), samarium (III), or ytterbium (III), holmium (III) or erbium (III) and the like. Based on especially pronounced magnetic moments, especially gadolinium (III), terbium (III), dysprosium (III), holmium (III) and erbium (III) are mostly preferred. Further one can select from radioisotopes. Examples of a few applicable radioisotopes include H 3, Be 10, O 15, Ca 49, Fe 60, In 111, Pb 210, Ra 220, Ra 224 and the like. Typically such ions are present as chelates or complexes, wherein for example as chelating agents or ligands for lanthanides and paramagnetic ions compounds such as diethylenetriamine pentaacetic acid (“DTPA”), ethylenediamine tetra acetic acid (“EDTA”), or tetraazacyclododecane-N,N′,N″,N′″-tetra acetic acid (“DOTA”) are used. Other typical organic complexing agents are for example published in Alexander, Chem. Rev. 95:273-342 (1995) and Jackels, Pharm. Med. Imag, Section III, Chap. 20, p 645 (1990). Other usable chelating agents in the present invention, are described in U.S. Pat. Nos. 5,155,215, 5,087,440, 5,219,553, 5,188,816, 4,885,363, 5,358,704, and 5,262,532, 5,188,816, 5,358,704, 4,885,363, and 5,219,553, and Meyer et al., Invest. Radiol. 25: S53 (1990). For example, salts and chelates can be used from the lanthanide group with the atomic numbers 57-83 or the transition metals with the atomic numbers 21-29, or 42 or 44.

It is possible to utilize paramagnetic perfluoroalkyl containing compounds which for example are described in German laid-open Patent Documents DE 196 03 033, DE 197 29 013 and in International Publication No. WO 97/26017, further diamagnetic perfluoroalkyl containing substances of the general formula R<PF>-L<II>-G<III>, wherein R<PF> represents a perfluoroalkyl group with 4 to 30 carbon atoms, L<II> stands for a linker and G<III> for a hydrophilic group. The linker L is a direct bond, an —SO₂— group or a straight or branched carbon chain with up to 20 carbon atoms which can be substituted with one or more —OH, —COO<−>, —SO₃-groups and/or if necessary one or more —O—, —S—, —CO—, —CONH—, —NHCO—, —CONR—, —NRCO—, —SO2-, —PO4-, —NH—, —NR-groups, an aryl ring or contain a piperazine, wherein R stands for a C₁ to C₂₀ alkyl group, which again can contain and/or have one or a plurality of O atoms and/or be substituted with —COO<−> or SO₃— groups.

The hydrophilic group G<III> can be selected from a mono or disaccharide, one or a plurality of —COO<−> or —SO₃<−>-groups, a dicarboxylic acid, an isophthalic acid, a picolinic acid, a benzenesulfonic acid, a tetrahydropyranedicarboxylic acid, a 2,6-pyridinedicarboxylic acid, a quaternary ammonium ion, an aminopolycarboxcylic acid, an aminodipolyethyleneglycol sulfonic acid, an aminopolyethyleneglycol group, an SO₂—(CH₂)₂—OH-group, a polyhydroxyalkyl chain with at least two hydroxyl groups or one or a plurality of polyethylene glycol chains having at least two glycol units, wherein the polyethylene glycol chains are terminated by an —OH or —OCH₃— group, or similar linkages. See for example published German patent DE 199 48 651, incorporated herein by reference in their entireties.

According to a further exemplary embodiment, it is possible to select paramagnetic metals in the form of metal complexes with phthalocyanines, especially as described in Phthalocyanine Properties and Applications, Vol. 14, C. C. Leznoff and A. B. P. Lever, VCH Ed., wherein as examples to mention are octa(1,4,7,10-tetraoxaundecyl)Gd-phthalocyanine, octa(1,4,7,10-tetraoxaundecyl)Gd-phthalocyanine, octa(1,4,7,10-tetraoxaundecyl)Mn-phthalocyanine, octa(1,4,7,10-tetraoxaundecyl)Mn-phthalocyanine, as described in U.S. 2004214810.

It is further possible to select from super-paramagnetic, ferromagnetic or ferrimagnetic signal generating agents. For example among magnetic metals, alloys may be preferable, among ferrites such as gamma iron oxide, magnetites or cobalt-, nickel- or manganese-ferrites, corresponding agents can be selected, e.g., particles as described in International Publication Nos. WO83/03920, WO83/01738, WO85/02772, WO89/03675, WO88/00060, WO90/01295 and WO90/01899, and U.S. Pat. Nos. 4,452,773, 4,675,173 and 4,770,183.

Further, magnetic, paramagnetic, diamagnetic or super paramagnetic metal oxide crystals having diameters of less than 4000 Angstroms are especially preferred as degradable non-organic agents. Suitable metal oxides can be selected from iron oxide, cobalt oxides, iridium oxides or the like, which provide suitable signal producing properties and which have especially biocompatible properties or are biodegradable. Mostly preferred are crystalline agents of this group having diameters smaller than 500 Angstroms. These crystals can be associated covalently or non-covalently with macromolecular species and are modified like the metal-based signal generating agents described above.

Further, zeolite containing paramagnets and gadolinium containing nanoparticles are selected from polyoxometallates, preferably of the lanthanides, (e.g., K9GdW10O36).

It is also possible to limit the average particle size of the magnetic signal producing agents to maximal 5 μm in order to optimize the image producing properties, and it is especially preferred that the magnetic signal producing particles be of a size from about 2 nm up to 1 μm, most preferably about 5 nm to 200 nm. The super paramagnetic signal producing agents can be chosen for example from the group of so-called SPIOs (super paramagnetic iron oxides) with a particle size larger than 50 nm or from the group of the USPIOs (ultra small super paramagnetic iron oxides) with particle sizes smaller than 50 nm.

In accordance with certain exemplary embodiments of the present invention, it can be preferred to select signal generating agents from the group of endohedral fullerenes, as described for example in U.S. Pat. No. 5,688,486 or International Publication No. WO 9315768, which are incorporated herein by reference in their entirety. It is possible to select fullerene derivatives and their metal complexes. Fullerene species can be used, which comprise carbon clusters having 60, 70, 76, 78, 82, 84, 90, 96 or more carbon atoms. An overview of such species described in European Patent Publication No 1331226A2, and incorporated herein by reference in its entirety.

Further metal fullerenes or endohedral carbon-carbon nanoparticles with arbitrary metal-based components can also be selected. Such endohedral fullerenes or endometallo fullerenes are particularly preferred, which for example contain rare earths such as cerium, neodymium, samarium, europium, gadolinium, terbium, dysprosium or holmium. Moreover it can be especially preferred to use carbon coated metallic nanoparticles such as carbides. The choice of nanomorphous carbon species is not limited to fullerenes, since it can be preferred to select from other nanomorphous carbon species such as nanotubes, onions, etc. In another embodiment it can be preferred to select fullerene species from non-endohedral or endohedral forms, which contain halogenated, preferably iodated, groups, as described in U.S. Pat. No. 6,660,248.

In certain embodiments mixtures of such signal generating agents of different specifications are also used, depending on the desired properties of the wanted signal generating material properties. The signal producing agents used generally can have a size of about 0.5 nm to 1000 nm, preferably about 0.5 nm to 900 nm, and further preferable from about 0.7 to 100 nm. In this connection the metal-based nanoparticles can be provided as a powder, in polar, non-polar or amphiphilic solutions, dispersions, suspensions or emulsions. Nanoparticles are easily modifiable based on their large surface to volume ratios. The nanoparticles to be selected can for example be modified non-covalently by means of hydrophobic ligands, for example with trioctylphosphine, or be covalently modified. Examples of covalent ligands are thiol fatty acids, amino fatty acids, fatty acid alcohols, fatty acids, fatty acid ester groups or mixtures thereof, for example oleic acid and oleylamine.

In accordance with exemplary embodiments of the present invention, the signal producing agents can be encapsulated in micelles or liposomes with the use of amphiphilic components, or may be encapsulated in polymeric shells, wherein the micelles/liposomes can have a diameter of about 2 nm to 800 nm, preferably from about 5 to 200 nm, and further preferable from about 10 to 25 nm. The size of the micelles/liposomes is, without committing to a specific theory, dependant on the number of hydrophobic and hydrophilic groups, the molecular weight of the nanoparticles and the aggregation number. In aqueous solutions the use of branched or unbranched amphiphilic substances, is especially preferred in order to achieve the encapsulation of signal generating agents in liposomes/micelles. The hydrophobic nucleus of the micelles hereby contains in a exemplary embodiment a multiplicity of hydrophobic groups, preferably between 1 and 200, especially preferred between 1 and 100 and mostly preferred between 1 and 30 according to the desired setting of the micelle size.

Hydrophobic groups consist preferably of hydrocarbon groups or residues or silicon-containing residues, for example polysiloxane chains. Furthermore, they can preferably be selected from hydrocarbon-based monomers, oligomers and polymers, or from lipids or phospholipids or comprise combinations hereof, especially glyceryl esters such as phosphatidyl ethanolamine, phosphatidyl choline, or polyglycolides, polylactides, polymethacrylate, polyvinylbutylether, polystyrene, polycyclopentadienylmethylnorbornene, polyethylenepropylene, polyethylene, polyisobutylene, polysiloxane. Further, for encapsulation in micelles hydrophilic polymers are also selected, especially preferred polystyrenesulfonic acid, poly-N-alkylvinylpyridiniumhalides, poly(meth)acrylic acid, polyamino acids, poly-N-vinylpyrrolidone, polyhydroxyethylmethacrylate, polyvinyl ether, polyethylene glycol, polypropylene oxide, polysaccharides such as agarose, dextrane, starches, cellulose, amylose, amylopectin, or polyethylene glycol or polyethylene imine of any desired molecular weight, depending on the desired micelles property. Further, mixtures of hydrophobic or hydrophilic polymers can be used or such lipid-polymer compositions employed. In a further special embodiment, the polymers are used as conjugated block polymers, wherein hydrophobic and also hydrophilic polymers or any desired mixtures there of can be selected as 2-, 3- or multi-block copolymers.

Such signal generating agents encapsulated in micelles can moreover be functionalized, while linker (groups) are attached at any desired position, preferably amino-, thiol, carboxyl-, hydroxyl-, succinimidyl, maleimidyl, biotin, aldehyde- or nitrilotriacetate groups, to which any desired corresponding chemically covalent or non-covalent other molecules or compositions can be bound according to the conventional. Here, especially biological molecules such as proteins, peptides, amino acids, polypeptides, lipoproteins, glycosaminoglycans, DNA, RNA or similar bio molecules are preferred especially.

It can be moreover preferred to select signal generating agents from non-metal-based signal generating agents, for example from the group of X-ray contrast agents, which can be ionic or non-ionic. Among the ionic contrast agents are included salts of 3-acetyl amino-2,4-6-triiodobenzoic acid, 3,5-diacetamido-2,4,6-triiodobenzoic acid, 2,4,6-triiodo-3,5-dipropionamido-benzoic acid, 3-acetyl amino-5-((acetyl amino)methyl)-2,4,6-triiodobenzoic acid, 3-acetyl amino-5-(acetyl methyl amino)-2,4,6-triiodobenzoic acid, 5-acetamido-2,4,6-triiodo-N-((methylcarbamoyl)methyl)-isophthalamic acid, 5-(2-methoxyacetamido)-2,4,6-triiodo-N-[2-hydroxy-1-(methylcarbamoyl)-ethoxy I]-isophthalamic acid, 5-acetamido-2,4,6-triiodo-N-methylisophthalamic acid, 5-acetamido-2,4,6-triiodo-N-(2-hydroxyethyl)-isophthalamic acid 2-[[2,4,6-triiodo-3[(1-oxobutyl)-amino]phenyl]methyl]-butanoic acid, beta-(3-amino-2,4,6-triiodophenyl)-alpha-ethyl-propanoic acid, 3-ethyl-3-hydroxy-2,4,6-triiodophenyl-propanoic acid, 3-[[(dimethylamino)-methyl]amino]-2,4,6-triiodophenyl-propanoic acid (see Chem. Ber. 93: 2347 (1960)), alpha-ethyl-(2,4,6-triiodo-3-(2-oxo-1-pyrrolidinyl)-phenyl)-propanoic acid, 2-[2-[3-(acetyl amino)-2,4,6-triiodophenoxy]ethoxymethyl]butanoic acid, N-(3-amino-2,4,6-triiodobenzoyl)-N-phenyl-.beta.-aminopropanoic acid, 3-acetyl-[(3-amino-2,4,6-triiodophenyl)amino]-2-methylpropanoic acid, 5-[(3-amino-2,4,6-triiodophenyl)methyl amino]-5-oxypentanoic acid, 4-[ethyl-[2,4,6-triiodo-3-(methyl amino)-phenyl]amino]-4-oxo-butanoic acid, 3,3′-oxy-bis[2,1-ethanediyloxy-(1-oxo-2,1-ethanediyl)imino]bis-2,4,6-triiodobenzoic acid, 4,7,10,13-tetraoxahexadecane-1,16-dioyl-bis(3-carboxy-2,4,6-triiodoanilide), 5,5′-(azelaoyldiimino)-bis[2,4,6-triiodo-3-(acetyl amino)methyl-benzoic acid], 5,5′-(apidoldiimino)bis(2,4,6-triiodo-N-methyl-isophthalamic acid), 5,5′-(sebacoyl-diimino)-bis(2,4,6-triiodo-N-methylisophthalamic acid), 5,5-[N,N-diacetyl-(4,9-dioxy-2,11-dihydroxy-1,12-dodecanediyl)diimino]bis(2,4,6-triiodo-N-methyl-isophthalamic acid), 5,5′5″-(nitrilo-triacetyltriimino)tris(2,4,6-triiodo-N-methyl-isophthalamic acid), 4-hydroxy-3,5-diiodo-alpha-phenylbenzenepropanoic acid, 3,5-diiodo-4-oxo-1(4H)-pyridine acetic acid, 1,4-dihydro-3,5-diiodo-1-methyl-4-oxo-2,6-pyridinedicarboxylic acid, 5-iodo-2-oxo-1(2H)-pyridine acetic acid, and N-(2-hydroxyethyl)-2,4,6-triiodo-5-[2,4,6-triiodo-3-(N-methylacetamido)-5-(methylcarbomoyl)benzamino]acetamido]-isophthalamic acid, and the like especially preferred, as well as other ionic X-ray contrast agents suggested in the literature, for example in J. Am. Pharm. Assoc., Sci. Ed. 42:721 (1953), Swiss Patent 480071, JACS 78:3210 (1956), German patent 2229360, U.S. Pat. No. 3,476,802, Arch. Pharm. (Weinheim, Germany) 306: 11 834 (1973), J. Med. Chem. 6: 24 (1963), FR-M-6777, Pharmazie 16: 389 (1961), U.S. Pat. Nos. 2,705,726 and 2,895,988, Chem. Ber. 93:2347 (1960), SA-A-68/01614, Acta Radiol. 12: 882 (1972), British Patent 870321, Rec. Trav. Chim. 87: 308 (1968), East German Patent 67209, German Patent 2050217, German Patent 2405652, Farm Ed. Sci. 28: 912 (1973), Farm Ed. Sci. 28: 996 (1973), J. Med. Chem. 9: 964 (1966), Arzheim.-Forsch 14: 451 (1964), SE-A-344166, British Patent No. 1346796, U.S. Pat. No. 2,551,696, U.S. Pat. Nos. 1,993,039, and 4,005,188, and Ann 494: 284 (1932), J. Pharm. Soc. (Japan) 50: 727 (1930).

Examples of applicable non-ionic X-ray contrast agents in accordance with the present invention, are metrizamide as described in German Patent publication 2031724, iopamidol as described in BE-A-836355, iohexyl as disclosed in Great Britain Patent Publication No. 1548594, iotrolan as described in European Patent Publication No. 33426, iodecimol as described in European Patent Publication No. 49745, iodixanol as described in European Patent Publication No. 108638, ioglucol as described in U.S. Pat. No. 4,314,055, ioglucomide as disclosed in BE-A-846657, ioglunioe as in DE-A-2456685, iogulamide as in BE-A-882309, iomeprol as described in European Patent Publication No. 26281, iopentol as described in European Patent Publication No. 105752, iopromide as described in European Patent Publication No. 2909439, iosarcol as described in German Patent Publication No. 3407473, iosimide as described in German Patent Publication No. 3001292, iotasul as in EP-A-22056, iovarsul as described in European Patent Publication No. 83964 or ioxilan in International Publication WO87/00757, and the like.

In certain exemplary embodiments, it is possible to select agents based on nanoparticle signal generating agents, which after release into tissues and cells are incorporated or are enriched in intermediate cell compartments and/or have an especially long residence time in the organism. Such particles are selected in a special embodiment from water-insoluble agents, in another embodiment, they contain a heavy element such as iodine or barium, in a third PH-50 as monomer, oligomer or polymer (iodinated aroyloxy ester having the empirical formula C₁₉H₂₃I₃N₂O₆, and the chemical names 6-ethoxy-6-oxohexy-3,5-bis(acetyl amino)-2,4,6-triiodobenzoate), in a particular exemplary embodiment an ester of diatrizoic acid, in a further exemplary embodiment of an iodinated aroyloxy ester or in a sixth embodiment any combinations hereof. In these embodiments particle sizes are preferred, which can be incorporated by macrophages. A corresponding method for this is disclosed in WO03039601 and agents preferred to be selected are disclosed in the publications U.S. Pat. Nos. 5,322,679, 5,466,440, 5,518,187, 5,580,579, and 5,718,388, gel of which are explicitly incorporated by reference. Especially advantageous are particularly, nanoparticles which are marked with signal generating agents or such signal generating agents such as PH-50, which accumulate in intercellular spaces and can make interstitial as well as extrastitial compartments visible.

Signal generating agents can be selected moreover from the group of the anionic or cationic lipids, as disclosed already in U.S. Pat. No. 6,808,720 and incorporated by reference in its entirety herewith. Exemplary are anionic lipids such as phosphatidyl acid, phosphatidyl glycerol and their fatty acid esters, or amides of phosphatidyl ethanolamine, such as anandamide and methanandamide, phosphatidyl serine, phosphatidyl inositol and their fatty acid esters, cardiolipin, phosphatidyl ethylene glycol, acid lysolipids, palmitic acid, stearic acid, arachidonic acid, oleic acid, linoleic acid, linolenic acid, myristic acid, sulfolipids and sulfatides, free fatty acids, both saturated and unsaturated and their negatively charged derivatives, and the like. Moreover, specially halogenated, in particular fluorinated anionic lipids are preferred. The anionic lipids preferably contain cations from the alkaline earth metals beryllium (Be<+2>), magnesium (Mg<+2>), calcium (Ca<+2>), strontium (Sr<+2>) and barium (Ba<+2>), or amphoteric ions, such as aluminium (Al<+3>), gallium (Ga<+3>), germanium (Ge<+3>), tin (Sn+<4>) or lead (Pb<+2> and Pb<+4>), or transition metals such as titanium (Ti<+3> and Ti<+4>), vanadium (V<+2> and V<+3>), chromium (Cr<+2> and Cr<+3>), manganese (Mn<+2> and Mn<+3>), iron (Fe<+2> and Fe<+3>), cobalt (Co<+2> and Co<+3>), nickel (Ni<+2> and Ni<+3>), copper (Cu<+2>), zinc (Zn<+2>), zirconium (Zr<+4>), niobium (Nb<+3>), molybdenum (Mo<+2> and Mo<+3>), cadmium (Cd<+2>), indium (In <+3>), tungsten (W<+2> and W<+4>), osmium (Os<+2>, Os<+3> and Os<+4>), iridium (Ir<+2>, Ir<+3> and Ir<+4>), mercury (Hg<+2>) or bismuth (Bi<+3>), and/or rare earths such as lanthanides, for example lanthanum (La<+3>) and gadolinium (Gd<+3>). Especially preferred cations are calcium (Ca<+2>), magnesium (Mg<+2>) and zinc (Zn<+2>) and paramagnetic cations such as manganese (Mn<+2>) or gadolinium (Gd<+3>).

Cationic lipids are to be selected from phosphatidyl ethanolamine, phospatidylcholine, Glycero-3-ethylphosphatidylcholine and their fatty acid esters, di- and tri-methylammoniumpropane, di- and tri-ethylammoniumpropane and their fatty acid esters. Especially preferred derivatives are N-[1-(2,3-dioleoyloxy)propyl]-N,N,N-trimethylammonium chloride (“DOTMA”); furthermore synthetic cationic lipids based on for example naturally occurring lipids such as dimethyldioctadecylammonium bromide, sphingolipids, sphingomyelin, lysolipids, glycolipids such as for example gangliosides GM1, sulfatides, glycosphingolipids, cholesterol and cholesterol esters or salts, N-succinyldioleoylphosphattidyl ethanolamine, 1,2,-dioleoyl-sn-glycerol, 1,3-dipalmitoyl-2-succinylglycerol, 1,2-dipalmitoyl-sn-3-succinylglycerol, 1-hexadecyl-2-palmitoylglycerophosphatidyl ethanolamine and palmitoyl-homocystein, mostly preferred are fluorinated, derivatized cationic lipids. Such compounds have been disclosed especially in U.S. Ser. No. 08/391,938.

Such lipids are furthermore suitable as components of signal generating liposomes, which especially can have pH-sensitive properties as described in U.S. Patent Publication No. 2004/197392.

In accordance with the exemplary embodiments of the present invention, signal generating agents can also be selected from the group of the so-called microbubbles or microballoons, which contain stable dispersions or suspensions in a liquid carrier substance. Gases to be chosen are preferably air, nitrogen, carbon dioxide, hydrogen or noble gases such as helium, argon, xenon or krypton, or sulfur-containing fluorinated gases such as sulfurhexafluoride, disulfurdecafluoride or trifluoromethylsulfurpentafluoride, or for example selenium hexafluoride, or halogenated silanes such as methylsilane or dimethylsilane, further short chain hydrocarbons such as alkanes, specifically methane, ethane, propane, butane or pentane, or cycloalkanes such as cyclopropane, cyclobutane or cyclopentane, also alkenes such as ethylene, propene, propadiene or butene, or also alkynes such as acetylene or propyne. Further ethers such as dimethylether can be considered or be chosen, or ketones, or esters or halogenated short-chain hydrocarbons or any desired mixtures of the above. Especially preferred are halogenated or fluorinated hydrocarbon gases such as bromochlorodifluoromethane, chlorodifluoromethane, dichlorodifluoromethan, bromotrifluoromethane, chlorotrifluoromethane, chloropentafluoroethane, dichlorotetrafluoroethane, chlorotrifluoroethylene, fluoroethylene, ethyl fluoride, 1,1-difluoroethane or perfluorohydrocarbons such as for example perfluoroalkanes, perfluorocycloalkanes, perfluoroalkenes or perfluorinated alkynes. Especially preferred are emulsions of liquid dodecafluoropentane or decafluorobutane and sorbitol, or similar, as disclosed in WO-A-93/05819 and explicitly incorporated herewith by reference.

Preferably, such microbubbles are selected, which are encapsulated in compounds having the structure R1-X-Z; R2-X-Z; or R3-X-Z′, wherein R1, R2 and R3 comprise hydrophobic groups selected from straight chain alkylenes, alkyl ethers, alkyl thiol ethers, alkyl disulfides, polyfluoroalkylenes and polyfluoroalkylethers, Z comprises a polar group from CO₂-M<+>, SO₃<−>M<+>, SO4<−>M<+>, PO₃<−>M<+>, PO₄<−>M<+2>, N(R)₄<+> or a pyridine or substituted pyridine, and a zwitterionic group, M is a metal ion, and finally X represents a linker which binds the polar group with the residues.

Gas-filled or in situ out-gassing micro spheres having a size of less than 1000 μm can be further selected from biocompatible synthetic polymers or copolymers which comprise monomers, dimers or oligomers or other pre-polymer to pre-stages of the following polymerizable substances: acrylic acid, methacrylic acid, ethyleneimine, crotonic acid, acryl amide, ethyl acrylate, methylmethacrylate, 2-hydroxyethylmethacrylate (HEMA), lactonic acid, glycolic acid, [epsilon]caprolactone, acrolein, cyanoacrylate, bisphenol A, epichlorhydrin, hydroxyalkylacrylate, siloxane, dimethylsiloxane, ethylene oxide, ethylene glycol, hydroxyalkylmethacrylate, N-substituted acryl amide, N-substituted methacrylamides, N-vinyl-2-pyrrolidone, 2,4-pentadiene-1-ol, vinyl acetate, acrylonitrile, styrene, p-aminostyrene, p-aminobenzylstyrene, sodium styrenesulfonate, sodium-2-sulfoxyethylmethacrylate, vinyl pyridine, aminoethylmethacrylate, 2-methacryloyloxytrimethylammonium chloride, and polyvinylidenes, such as polyfunctional cross-linkable monomers such as for example N,N′-methylene-bis-acrylamide, ethylene glycol dimethacrylate, 2,2′-(p-phenylenedioxy)-diethyldimethacrylate, divinylbenzene, triallylamine and methylene-bis-(4-phenyl-isocyanate), including any desired combinations thereof. Preferred polymers contain polyacrylic acid, polyethyleneimine, polymethacrylic acid, polymethylmethacrylate, polysiloxane, polydimethylsiloxane, polylactonic acid, poly([epsilon]-caprolactone), epoxy resins, poly(ethylene oxide), poly(ethylene glycol), and polyamides (e.g. Nylon) and the like or any arbitrary mixtures thereof. Preferred copolymers contain among others polyvinylidene-polyacrylonitrile, polyvinylidene-polyacrylonitrile-polymethylmethacrylate, and polystyrene-polyacrylonitrile and the like or any desired mixtures thereof. Methods for manufacture of such micro spheres are published for example in Garner et al., U.S. Pat. Nos. 4,179,546, 3,945,956, 4,108,806, Japan Kokai Tokkyo Koho 62 286534, British Patent No. 1,044,680, Kenaga et al., U.S. Pat. Nos. 3,293,114, 3,401,475, 3,479,811, 3,488,714, 3,615,972, 4,549,892, 4,540,629, 4,421,562, 4,420,442, 4,898,734, 4,822,534, 3,732,172, 3,594,326, 3,015,128, Deasy, Microencapsulation and Related Drug Processes, Vol. 20, Chapters. 9 and 10, pp. 195-240 (Marcel Dekker, Inc., N.Y., 1984), Chang et al., Canadian J of Physiology and Pharmacology, Vol 44, pp. 115-129 (1966), and Chang, Science, Vol. 146, pp. 524-525 (1964).

Other exemplary signal generating agents can in accordance with the present invention be selected from the group of agents, which are transformed into signal generating agents in organisms by means of in-vitro or in-vivo cells, cells as a component of cell cultures, of in-vitro tissues, or cells as a component of multicellular organisms, such as for example fungi, plants or animals, in exemplary embodiments from mammals such as mice or humans. Such agents can be made available in the form of vectors for the transfection of multicellular organisms, wherein the vectors contain recombinant nucleic acids for the coding of signal generating agents. In certain embodiments this is concerned with signal generating agents such as metal binding proteins. It can be preferred to choose such vectors from the group of viruses for example from adeno viruses, adeno virus associated viruses, herpes simplex viruses, retroviruses, alpha viruses, pox viruses, arena-viruses, vaccinia viruses, influenza viruses, polio viruses or hybrids of any of the above.

Further such signal generating agents are to be chosen in combination with delivery systems, in order to incorporate nucleic acids, which are suitable for coding for signal generating agents, into the target structure. Especially preferred are virus particles for the transfection of mammalian cells, wherein the virus particle contains one or a plurality of coding sequence/s for one or a plurality of signal generating agents as described above. In these cases the particles are generated from one or a plurality of the following viruses: adeno viruses, adeno virus associated viruses, herpes simplex viruses, retroviruses, alpha viruses, pox viruses, arena-viruses, vaccinia viruses, influenza viruses and polio viruses.

In further exemplary embodiments, these signal generating agents are made available from colloidal suspensions or emulsions, which are suitable to transfect cells, preferably mammalian cells, wherein these colloidal suspensions and emulsions contain those nucleic acids which possess one or a plurality of the coding sequence(s) for signal generating agents. Such colloidal suspensions or emulsions can contain macromolecular complexes, nano capsules, microspheres, beads, micelles, oil-in-water- or water-in-oil emulsions, mixed micelles and liposomes or any desired mixture of the above.

In further embodiments, cells, cell cultures, organized cell cultures, tissues, organs of desired species and non-human organisms can be chosen which contain recombinant nucleic acids having coding sequences for signal generating agents. In specific embodiments organisms are selected from the groups: mouse, rat, dog, monkey, pig, fruit fly, nematode worms, fish or plants or fungi. Further, cells, cell cultures, organized cell cultures, tissues, organs of desired species and non-human organisms can contain one or a plurality of vectors as described above.

Signal generating agents can preferably be produced in vivo from the group of proteins and made available as described above. Such agents can be preferably directly or indirectly signal producing, while the cells produce (direct) a signal producing protein through transfection or produce a protein which induces (indirect) the production of a signal producing protein. Preferably these signal generating agents are detectable in methods such as MRI while the relaxation times T1, T2 or both are altered and lead to signal producing effects which can be processed sufficiently for imaging. Such proteins are preferably protein complexes, especially metalloprotein complexes. Direct signal producing proteins are preferably such metalloprotein complexes which are formed in the cells. Indirect signal producing agents are such proteins or nucleic acids, for example, which regulate the homeostasis of iron metabolism, the expression of endogenous genes for the production of signal generating agents, and/or the activity of endogenous proteins with direct signal generating properties, for example Iron Regulatory Protein (IRP), Transferrin receptor (for the take-up of Fe), erythroid-5-aminobevulinate synthase (for the utilization of Fe, H-Ferritin and L-Ferritin for the purpose of Fe storage). In specific embodiments it can be preferred to combine both types of signal generating agents, that is direct and indirect, with each other, for example an indirect signal generating agent, which regulates the iron-homeostasis and a direct agent, which represents a metal binding protein.

In such exemplary embodiments, where preferably metal-binding polypeptides are selected as indirect agents, it is advantageous if the polypeptide binds to one or a plurality of metals which possess signal generating properties. Especially preferred are such metals with unpaired electrons in the Dorf orbitals, such as for example Fe, Co, Mn, Ni, Gd etc., wherein especially Fe is available in high physiological concentrations in organisms. It is moreover preferred, if such agents form metal-rich aggregates, for example crystalline aggregates, whose diameters are larger than 10 picometers, preferably larger than 100 picometers, 1 nm, 10 nm or specially preferred larger than 100 nm.

It is possible to use such metal-binding compounds, which have sub-nanomolar affinities with dissociation constants of less than 10⁻¹⁵ M, 10⁻² M or smaller. Typical polypeptides or metal-binding proteins are lactoferrin, ferritin, or other dimetallocarboxylate proteins or the like, or so-called metal catcher with siderophoric groups, such as for example haemoglobin. A possible method for preparation of such signal generating agents, their selection and the possible direct or indirect agents which are producible in vivo and are suitable as signal generating agents was disclosed in WO 03/075747 and is incorporated herewith in accordance with the present invention.

Another group of signal generating agents can be photophysically signal producing agents which consist of dyestuff-peptide-conjugates. Such dyestuff-peptide-conjugates are preferred which provide a wide spectrum of absorption maxima, for example polymethin dyestuffs, in particular cyanine-, merocyanine-, oxonol- and squarilium dyestuffs. From the class of the polymethin dyestuffs the cyanine dyestuffs, e.g. the indole structure based indocarbo-, indodicarbo- and indotricarbocyanines, are especially preferred. Such dyestuffs can be preferred in specific embodiments, which are substituted with suitable linking agents and can be functionalized with other groups as desired. In this connection see also German Publication 19917713.

In accordance with certain exemplary embodiments of the present invention, signal generating agents can be functionalized as desired. The functionalization by means of so-called “Targeting” groups is preferred are to be understood, as functional chemical compounds which link the signal generating agent or its specifically available form (encapsulation, micelles, micro spheres, vectors etc.) to a specific functional location, or to a determined cell type, tissue type or other desired target structures. Preferably targeting groups permit the accumulation of signal-producing agents in or at specific target structures. Therefore the targeting groups can be selected from such substances, which are principally suitable to provide a purposeful enrichment of the signal generating agents in their specifically available form by physical, chemical or biological routes or combinations thereof. Useful targeting groups to be selected can therefore be antibodies, cell receptor ligands, hormones, lipids, sugars, dextrane, alcohols, bile acids, fatty acids, amino acids, peptides and nucleic acids, which can be chemically or physically attached to signal-generating agents, in order to link the signal-generating agents into/onto a specifically desired structure. In a first embodiment targeting groups are selected, which enrich signal-generating agents in/on a tissue type or on surfaces of cells. Here it is not necessary for the function, that the signal generating agent be taken up into the cytoplasm of the cells. Peptides are preferred as targeting groups, for example chemotactic peptides are used to make inflammation reactions in tissues visible by means of signal generating agents; in this connection see also WO 97/14443.

Antibodies can also be preferred, including antibody fragments, Fab, Fab2, Single Chain Antibodies (for example Fv), chimerical antibodies, and the like, as known from the conventional, moreover antibody-like substances, for example so-called anticalines, wherein it is unimportant whether the antibodies are modified after preparation, recombinants are produced or whether they are human or non-human antibodies. It is preferred to choose from humanized or human antibodies, examples of humanized forms of non-human antibodies are chimerical immunoglobulines, immunoglobulin chains or fragments (such as Fv, Fab, Fab′, F(ab″)2 or other antigen-binding subsequences of antibodies, which partly contain sequences of non-human antibodies; humanized antibodies contain for example human immunoglobulines (receptor or recipient antibody), in which groups of a CDR (Complementary Determining Region) of the receptor are replaced through groups of a CDR of a non-human (spender or donor antibody), wherein the spender species for example, mouse, rabbit or other has appropriate specificity, affinity, and capacity for the binding of target antigens. In a few forms the Fv framework groups of the human immunglobulines are replaced by means of corresponding non-human groups. Humanized antibodies can moreover contain groups which either do not occur in either the CDR or Fv framework sequence of the spender or the recipient. Humanized antibodies essentially comprise substantially at least one or preferably two variable domains, in which all or substantial components of the CDR components of the CDR regions or Fv framework sequences correspond with those of the non-human immunoglobulin, and all or substantial components of the FR regions correspond with a human consensus-sequence. In accordance with the present invention targeting groups of this embodiment can also be hetero-conjugated antibodies. Preferred function of the selected antibodies or peptides are cell surface markers or molecules, particularly of cancer cells, wherein here a large number of known surface structures are known, such as HER2, VEGF, CA15-3, CA 549, CA 27.29, CA 19, CA 50, CA242, MCA, CA125, DE-PAN-2, etc., and the like.

Moreover, it may be preferred to select targeting groups which contain the functional binding sites of ligands. Such can be chosen from all types, which are suitable for binding to any desired cell receptors. Examples of possible target receptors are, without limiting the choice, receptors of the group of insulin receptors, insulin-like growth factor receptor (e IGF-1 and IGF-2), growth hormone receptor, glucose transporters (particularly GLUT 4 receptor), transferrin receptor (transferrin), Epidermal Growth Factor receptor (EGF), low density lipoprotein receptor, high density lipoprotein receptor, leptin receptor, oestrogen receptor; interleukin receptors including IL-1, IL-2, IL-3, IL-4, IL-5, IL-6, IL-7, IL-8, IL-9, IL-11, IL-12, IL-13, IL-15, and IL-17 receptor, VEGF receptor (VEGF), PDGF receptor (PDGF), Transforming Growth Factor receptor (including TGF-[alpha] and TGF-[beta]), EPO receptor (EPO), TPO receptor (TPO), ciliary neurotrophic factor receptor, prolactin receptor, and T-cell receptors.

It can be preferred to select hormone receptors, especially for hormones such as steroidal hormones or protein- or peptide-based hormones, for example, however not limited thereto, epinephrines, thyroxines, oxytocine, insulin, thyroid-stimulating hormone, calcitonine, chorionic gonadotropine, corticotropine, follicle stimulating hormone, glucagons, leuteinizing hormone, lipotropine, melanocyte-stimulating hormone, norepinephrines, parathyroid hormone, Thyroid-Stimulating Hormone (TSH), vasopressin's, encephalin, serotonin, estradiol, progesterone, testosterone, cortisone, and glucocorticoide. Receptor ligands include those which are on the cell surface receptors of hormones, lipids, proteins, glycol proteins, signal transducers, growth factors, cytokine, and other bio molecules. Moreover, targeting groups can be selected from carbohydrates with the general formula: Cₓ(H₂O)_{y}, wherein herewith also monosaccharides, disaccharides and oligo- as well as polysaccharides are included, as well as other polymers which consist of sugar molecules which contain glycosidic bonds. Specially preferred carbohydrates are those in which all or parts of the carbohydrate components contain glycosylated proteins, including the monomers and oligomers of galactose, mannose, fructose, galactosamine, glucosamine, glucose, sialic acid, and especially the glycosylated components, which make possible the binding to specific receptors, especially cell surface receptors. Other useful carbohydrates to be selected contain monomers and polymers of glucose, ribose, lactose, raffinose, fructose and other biologically occurring carbohydrates especially polysaccharides, for example, however not exclusively, arabinogalactan, gum Arabica, mannan and the like, which are usable in order to introduce signal generating agents into cells. Reference is made in this connection to U.S. Pat. No. 5,554,386.

Furthermore, targeting groups can be selected from the lipid group, wherein also fats, fatty oils, waxes, phospholipids, glycolipids, terpenes, fatty acids and glycerides, especially triglycerides are included. Further included are eicosanoides, steroids, sterols, suitable compounds of which can also be hormones such as prostaglandins, opiates and cholesterol and the like. In accordance with the present invention, all functional groups can be selected as the targeting group, which possess inhibiting properties, such as for example enzyme inhibitors, preferably those which link signal generating agents into/onto enzymes.

In another exemplary embodiment, targeting groups can be selected from a group of functional compounds which make possible internalization or incorporation of signal generating agents in the cells, especially in the cytoplasm or in specific cell compartments or organelles, such as for example the cell nucleus. For example targeting group is preferred which contains all or parts of HIV-1 tat-proteins, their analogs and derivatized or functionally similar proteins, and in this way allows an especially rapid uptake of substances into the cells. As an example refer to Fawell et al., PNAS USA 91:664 (1994); Frankel et al., Cell 55:1189, (1988); Savion et al., J. Biol. Chem. 256:1149 (1981); Derossi et al., J. Biol. Chem. 269:10444 (1994); and Baldin et al., EMBO J. 9:1511 (1990).

Targeting groups can be further selected from the so-called Nuclear Localisation Signal (NLS), where under short positively charged (basic) domains are understood which bind to specifically targeted structures of cell nuclei. Numerous NLS and their amino acid sequences are known including single basic NLS such as that of the SV40 (monkey virus) large T Antigen (pro Lys Lys Lys Arg Lys Val), Kalderon (1984), et al., Cell, 39:499-509), the teinoic acid receptor-[beta] nuclear localization signal (ARRRRP); NFKB p50 (EEVQRKRQKL; Ghosh et al., Cell 62:1019 (1990); NFKB p65 (EEKRKRTYE; Nolan et al., Cell 64:961 (1991), as well as others (see for example Boulikas, J. Cell. Biochem. 55(1):32-58 (1994), and double basic NLS's such as for example xenopus (African clawed toad) proteins, nucleoplasmin (Ala Val Lys Arg Pro Ala Ala Thr Lys Lys Ala Gly Gln Ala Lys Lys Lys Lys Leu Asp), Dingwall, et al., Cell, 30:449-458, 1982 and Dingwall, et al., J. Cell Biol., 107:641-849, 1988. These are all incorporated herewith by reference in accordance with the present invention. Numerous localization studies have shown that NLSs, which are built into synthetic peptides which normally do not address the cell nucleus or were coupled to reporter proteins, lead to an enrichment of such proteins and peptides in cell nuclei. In this connection exemplary references are made to Dingwall, and Laskey, Ann, Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990. It can be especially preferred to select targeting groups for the hepatobiliary system, wherein in U.S. Pat. Nos. 5,573,752 and 5,582,814 corresponding groups are suggested.

In certain exemplary embodiments, the implant comprises absorptive agents, e.g. to remove compounds from body fluids. Suitable absorptive agents, but not exclusively and not limited to, are chelating agents such as penicillamine, methylene tetramine dihydrochloride, EDTA, DMSA or deferoxamine mesylate, any other appropriate chemical modification of the coating surface, antibodies, and microbeads or other materials containing cross linked reagents for absorption of drugs, toxins or other agents.

In some specifically exemplary embodiments biologically active agents are selected from cells, cell cultures, organized cell cultures, tissues, organs of desired species and non-human organisms.

In further exemplary embodiments, the beneficial agents comprise metal based nano-particles that are selected from ferromagnetic or superparamagnetic metals or metal-alloys, either further modified by coating with silanes or any other suitable polymer or not modified, for interstitial hyperthermia or thermoablation. In further embodiments, the exemplary compositions can comprise silver nano-particles or other anti-infective inorganic materials, preferably as nano-particles with a D50 between 10 nm and 50 nm, whereby the amount of the anti-infective particles is at least 1 weight %, preferably 2-5 weight % and more preferred 5 to 10 weight %, most preferred between 10 and 20 weight %.

The exemplary embodiments of the present invention is now further described by the following examples, which do not limit the present invention to particular details mentioned therein.

### EXAMPLES

### Example 1

### Preparation of a Curable Matrix

5.0 g of triethylene glycol dimethacrylate and 4-methoxyphenol (min. 95%, available from PolySciences Inc.) were mixed at room temperature with 0.2 g of bisphenol-A-dimethacrylate (available from Sigma-Aldrich) and 0.5 g of N,N′-dihydroxy-ethyl-p-toluidine (available from Sigma-Aldrich) in a conventional glass beaker equipped with a magnetic stirrer at approximately 50 rpm for 30 minutes to obtain a homogeneous solution. Subsequently, 12 g of ethyoxylated bisphenol-A-dimethacrylate (available from Sigma-Aldrich) with butylated hydroxytoluene (Sigma-Aldrich) and 16 g of Bis-GMA (Bisphenol-A-Glycidin-dimethacrylat, Sigma-Aldrich) were added to the solution and mixed for 60 minutes.

### Example 2

### Preparation of Catalyst for Curing

10 g of triethylene glycol dimethacrylate (purum, available from Sigma Aldrich), 0.05 gram of butylated hydroxytoluene (Sigma-Aldrich) and 1 g of benzoyl peroxide (purum, moistened with water, ≧97.0%, available from Sigma-Aldrich) were mixed together until a uniform solution was obtained (room temperature, 50 rpm). Then, 30 g of BIS-GMA (Sigma-Aldrich) was added and stirred for approximately 60 minutes.

### Example 3

### Preparation of a Magnesium Cement Composite

0.4 g of Magnesium powder (available from Goodfellow) with an average particle size of 250 μm (purity >99.8%) was manually mixed for 5 minutes with 5 g of the curable matrix of Example 1 to a grey dispersion in a glass dish. Then 5 g of the catalyst of Example 2 was added and manually mixed using a glass spatula for approximately 50 seconds. The obtained mixture had a dark grey colour. Subsequently, a cross-linking reaction was observed with a slight warming of the mixture over a period of approximately 4 to 6 minutes. The obtained composite was hardened to obtain a partially degradable implant material (by biodegradation of magnesium).

### Example 4

### Light-Curable Cement

1 g of ethyl dimethylaminobenzoate (>99%, Sigma-Aldrich) was added to 40 g of a mixture of magnesium hydroxide powder (<100 nm particle size, Sigma-Aldrich) and magnesium powder (as specified in Example 3 above) (w/w 1:1) and gently mixed to give a cement powder. A matrix material was produced by dissolving 10 g of polyacrylic acid (average MW 450.000, Sigma-Aldrich) in 40 g of 2-hydroxyethyl methacrylate (97%, Sigma-Aldrich). Subsequently, 0.4 g of camphorquinone (a photo-sensitizer, 97%, available from Sigma-Aldrich) was added and stirred to a homogeneous liquid. Afterwards, 5 g of the cement powder was mixed with 1.5 g of the liquid and kneaded for 50 seconds. The resulting mixture can be handled for at least 40 Minutes without substantial curing. Subsequently, the paste was molded in cylindrical test molds and treated with a standard industrial visible irradiation polymerizer for curing, to provide a test implant being partially degradable by degradation of the magnesium components.

### Example 5

### Degradable Bone Implant and Cement Material

Poly-(propylene fumarate) was synthesized from diethyl fumarate (purum, >97%, available from Sigma-Aldrich) and propylene glycol (99%, available from Sigma-Aldrich) by transesterification as described in U.S. Pat. No. 4,722,948. A moldable, curable paste was prepared by mixing 6 g MgO (≧95%, fused, 150-325 mesh, available from Sigma-Aldrich), 6 g magnesium powder (Goodfellow), 10 g poly-(propylene fumarate) and 3 g N-vinyl-2-pyrrolidone (>99%, Sigma-Aldrich). Separately, a cross-linking mixture was produced by mixing 0.5 g benzoyl peroxide dissolved in 5 g N-vinyl-2-pyrrolidone. After mixing of paste and cross-linking the mixture the resulting cement remains moldable for approximately 20 to 35 minutes after which the structure remains cured and stable. The resulting material is partially biodegradable by degrading of magnesium.

While magnesium-based degradable metallic materials have been used in the above illustrative examples, all other degradable materials as described herein are equally suitable instead of the magnesium, as the skilled person is well aware of. Also, the polymer matrix may be selected from any of the materials described herein, particularly also from degradable matrix materials.

It should be noted that the term ‘comprising’ does not exclude other elements or steps and the ‘a’ or ‘an’ does not exclude a plurality. In addition elements described in association with the different embodiments may be combined.

Having thus described in detail several exemplary embodiments of the present invention, it is to be understood that the present invention described above is not to be limited to particular details set forth in the above description, as many apparent variations thereof are possible without departing from the spirit or scope of the present invention. The exemplary embodiments of the present invention are disclosed herein or are obvious from and encompassed by the detailed description. The detailed description, given by way of example, but not intended to limit the present invention solely to the specific embodiments described.

The foregoing applications, and all documents cited therein or during their prosecution (“appln. cited documents”) and all documents cited or referenced in the appln. cited documents, and all documents cited or referenced herein (“herein cited documents”), and all documents cited or referenced in the herein cited documents, together with any manufacturer's instructions, descriptions, product specifications, and product sheets for any products mentioned herein or in any document incorporated by reference herein, are hereby incorporated herein by reference, and may be employed in the practice of the present invention.

What is claimed is:

1. A curable therapeutic implant composition for use in the filling of a cavity in a living organism, comprising
a) particles of a metallic material, and
b) a curable matrix-forming a non-particulate material,
wherein at least one of the metallic material or the matrix-forming material is at least partially degradable in-vivo.

2. The composition of claim 1, wherein the metallic material includes at least one of a metal or a metal alloy.

3. The composition of claim 1, wherein the metallic material particles are degradable in-vivo.

4. The composition of claim 2, wherein the degradable metal or alloy includes at least one of an alkaline metal, an alkaline earth metal, Fe, Zn, Al, Mg, Ca, Zn, W, Ln, Si, or Y.

5. The composition of claim 4, wherein the degradable metallic material is combined with other metallic particles which include at least one of Mn, Co, Ni, Cr, Cu, Cd, Pb, Sn, Th, Zr, Ag, Au, Pd, Pt, Si, Ca, Li, Al, Zn or Fe.

6. The composition of claim 4, wherein the degradable metallic material includes a magnesium alloy comprising more than 90% of Mg, about 4-5% of Y, and about 1.5-4% of other rare earth metals.

7. The composition of claim 4, wherein the degradable metallic material particles comprises a metal alloy of at least one of:
(i) 10-98 wt.-%, such as 35-75 wt.-% of Mg, and 0-70 wt.-%, such as 30-40% of Li and 0-12 wt.-% of other metals,
(ii) 60-99 wt.-% of Fe, 0.05-6 wt.-% Cr, 0.05-7 wt.-% Ni and up to 10 wt.-% of other metals; or
(iii) 60-96 wt.-% Fe, 1-10 wt.-% Cr, 0.05-3 wt.-% Ni and 0-15 wt.-% of other metals,
wherein individual weight ranges are selected to sum to about 100 wt.-% in total for each alloy.

8. The composition of claim 2, wherein the metallic material particles are substantially non-degradable in-vivo.

9. The composition of claim 8, wherein the metallic material includes at least one metal of main group metals of the periodic system, transition metals such as copper, gold and silver, titanium, zirconium, hafnium, vanadium, niobium, tantalum, chromium, molybdenum, tungsten, manganese, rhenium, iron, cobalt, nickel, ruthenium, rhodium, palladium, osmium, iridium or platinum, or rare earth metals.

10. The composition of claim 2, wherein the metallic material includes a biocorrosive alloy which includes biocorrosive alloys comprising as a major component one of tungsten, rhenium, osmium or molybdenum.

11. The composition of claim 10, wherein the biocorrosive alloy further comprises one of cerium, an actinide, iron, tantalum, platinum, gold, gadolinium, yttrium or scandium.

12. The composition of claim 2, wherein the metallic material particles comprise a mixture of at least one first metallic material and at least one second metallic material, the first metallic material being more electronegative than the second metallic material, such that the first and second metallic material particles form a local cell at their contact surfaces.

13. The composition of claim 1, wherein an average particle size of the metallic material is from about 0.5 nm to about 5000 μm.

14. The composition of claim 1, wherein the curable matrix-forming a non-particulate material is an organic material comprising a polymer or a polymer-solvent system.

15. The composition of claim 14, wherein the polymer-solvent system is a mixture of at least one polymer and at least one solvent or plasticizer.

16. The composition of claim 14, wherein the organic material comprises at least one of an oligomer, polymer or copolymer including at least one of a poly(meth)acrylate, unsaturated polyester, saturated polyester, polyolefines, polyethylene, polypropylene, polybutylene, alkyd resins, epoxy-polymers or resins, polyamide, polyimide, polyetherimide, polyamideimide, polyesterimide, polyester amide imide, polyurethane, polycarboxylate, polycarbonate, polystyrene, polyphenol, polyvinyl ester, polysilicone, polyacetal, cellulosic acetate, polyvinylchloride, polyvinyl acetate, polyvinyl alcohol, polysulfone, polyphenylsulfone, polyethersulfone, polyketone, polyetherketone, polybenzimidazole, polybenzoxazole, polybenzothiazole, polyfluorocarbons, polyphenylene ether, polyarylate, or cyanatoester-polymers, and any of the copolymers and any mixtures thereof.

17. The composition of claim 14, wherein the organic material comprises one of a polymer or copolymer selected from at least one of collagen, albumin, gelatin, hyaluronic acid, starch, cellulose, methylcellulose, hydroxypropylcellulose, hydroxypropylmethyl-cellulose, carboxymethylcellulose-phthalate; gelatine, casein, dextrane, polysaccharide, fibrinogen, poly(D,L lactide), poly(D,L-lactide-co-glycolide), poly(glycolide-co-trimethylene carbonates), poly(glycolide), poly(hydroxybutylate), poly(alkylcarbonate), poly(a-hydroxyesters), poly(ether esters, poly(orthoester), polyester, poly(hydroxyvaleric acid), polydioxanone, poly(ethylene terephtalate), poly(maleic acid), poly(malic acid), poly(tartaric acid), polyanhydride, polyphosphazene, poly(amino acids), polypeptides, polycaprolactones, poly(propylene fumarates), poly(ester amides), poly(ethylene fumarates), poly(hydroxy butyrates), and polyurethanes.

18. The composition of claim 14, wherein the organic material is at least partially biodegradable.

19. The composition of claim 14, wherein the solvent is added in an amount sufficient to soften the polymer but not liquefy the polymer.

20. The composition of claim 19, wherein the composition is in the form of a viscous paste having a viscosity from about 200 Pa·s to 800 Pa·s.

21. The composition of claim 14, wherein a sufficient quantity of liquid solvent is added to the polymer to liquefy at least a part of the polymer.

22. The composition of claim 14, wherein the plasticizer is a solvent that has solubility in an aqueous medium, ranging from miscible to dispersible.

23. The composition of claim 14, wherein a solvent or a plasticizer includes at least one of water, an alcohol, acetone, ethyl lactate, ethyl acetate, ethyl acetoacetate, ethyl formate, acetyltributylcitrate, triethyl citrate, tetrahydrofuran, toluene, and n-methyl-2-pyrrolidone (NMP).

24. The composition of claim 14, wherein the is curable by extraction of the solvent or plasticizer.

25. The composition of claim 24, wherein the extraction involves a diffusion of the solvent or plasticizer into an aqueous medium ex-vivo, or into body fluids in-vivo, or removal of the solvent or plasticizer by one of drying, freeze-drying, or evaporation.

26. The composition of claim 1, wherein the curable matrix-forming a non-particulate material is an organic material comprising at least one polymerizable or crosslinkable monomer, selected from at least one of a monofunctional monomer or a polyfunctional monomer.

27. The composition of claim 26, wherein the monofunctional monomer includes at least one of methyl acrylate, methyl methacrylate, ethyl acrylate, ethyl methacrylate, butyl acrylate, butyl methacrylate, acryl acrylate, acryl methacrylate, hydroxyethyl acrylate, hydroxyethyl methacrylate, methoxyethyl acrylate, or methoxyethyl methacrylate.

28. The composition of claim 26, wherein the polyfunctional monomer includes at least one of bifunctional aliphatic acrylates, bifunctional aliphatic methacrylates, bifunctional aromatic acrylates, bifunctional aromatic methacrylates, trifunctional aliphatic acrylates, trifunctional aliphatic methacrylates, tetrafunctional acrylates, and tetrafunctional methacrylates, such as triethylene glycol diacrylate, triethylene glycol dimethacrylate, 2,2-bis(4-methacryloxyphenyl)propane, 2,2-bis(4-methacryloxyethoxyphenyl)propane, 2,2-bis(4-methacryloxypolyethoxyphenyl]-propane, 2,2-bis[4-(3-methacryloxy-2-hydroxypropoxy)-phenyl]propane, di(methacryloxyethyl)trimethylhexamethylene diurethane, tetramethylolmethane tetraacrylate, or tetramethylolmethane tetramethacrylate.

29. The composition of claim 26, wherein the polyfunctional monomer includes at least one of a di(meth)acrylate, such as urethane dimethacrylate, ethyleneglycol dimethacrylate, (2,2-bis[4-(2-hydroxy-3-methacryloxypropoxy)-phenyl]propane (BIS-GMA), (2,2-bis[4-(methacryloxy)phenyl]propane (BIS-MA), 2,2-bis(4-methacryloxyphenyl)propane, 1,2-butanediol-diacrylate, 1,4-butanediol-diacrylate, 1,4-butanediol-dimethacrylate, 1,4-cyclo-hexanediol-dimethacrylate, 1,10-decanediol-dimethacrylate, diethylene-glycol-diacrylate, dipropyleneglycol-diacrylate, dimethylpropanediol-dimethacrylate, triethyleneglycol-dimethacrylate (TEGDMA), tetraethyleneglycol-dimethacrylate, 1,6-hexanediol-diacrylate, 1,6-bis-[2-methacryloxyethoxycarbonylamino]-2,2,4-trimethylhexane (UDMA), neopentylglycol-diacrylate, polyethyleneglycol-dimethacrylate, tripropyleneglycol-diacrylate, 2,2-bis-[4-(2-acryloxyethoxy)phenyl]propane, 2,2-bis-[4-(2-hydroxy-3-methacryloxypropoxy)phenyl]propane, bis(2-methacryloxyethyl)N,N-1,9-nonylene-biscarbamate, 1,4-cyclohexanedimethanol-dimethacrylate, or diacrylic urethane oligomers.

30. The composition of claim 26, further comprising at least one of a polymerization catalyst, an initiator, or an accelerator.

31. The composition of claim 30, wherein the catalyst is a photoinitiator including camphorquinone, an accelerator including ethyl-p-dimethylaminobenzoate (DMAB) or N,N-dimethylaminoethyl methacrylate (DMAEMA), or a redox catalyst, including at least one of a combination of an amine and a peroxide, a combination of a sulfinic acid and a peroxide, or a combination of an other material and a peroxide.

32. The composition of claim 31, wherein the peroxide includes at least one of a diacyl peroxide including at least one of benzoyl peroxide, p-chlorobenzoyl peroxide, 2,4-dichlorobenzoyl peroxide, acetyl peroxide, and lauroyl peroxide; or a hydroperoxide such as t-butyl hydroperoxide, cumene hydroperoxide, and 2,5-dimethylhexane 2,5-dihydroperoxide; or a ketone peroxide including at least one of methyl ethyl ketone peroxide; or a peroxycarbonate.

33. The composition of claim 31, wherein the amine includes at least one of N,N-bis-(2-hydroxyethyl)4-methylaniline, N,N-bis-(2-hydroxyethyl)-3,4-dimethylaniline, N,N-bis-(2-hydroxyethyl)-3,5-dimethylaniline, N-methyl-N-(2-hydroxyethyl)-4-methylaniline, 4-methylaniline, N,N-dimethyl-p-toluidine (DMPT), N,N-dimethylaniline, or triethanolamine.

34. The composition of claim 31, wherein the sulfinic acid includes at least one of p-toluenesulfinic acid, benzenesulfinic acid, and salts thereof.

35. The composition of claim 31, wherein an other material which is combined with the peroxide includes at least one of cobalt naphthenate, cobalt octanate, trimethyl barbituric acid, and a trialkyl boron.

36. The composition of claim 31, wherein two component system is provided, wherein the composition is divided into two parts, the amine or sulfinic acid is incorporated into one part whereas the peroxide is incorporated into the other part, and the both parts are to be mixed at the time of use.

37. The composition of claim 1, wherein the curable matrix-forming, non-particulate material includes precursor compounds of an inorganic-organic hybrid material, processible by sol-gel processing.

38. The composition of claim 37, wherein the sol-gel-processing involves one of hydrolytic or non-hydrolytic sol-gel processing.

39. The composition of claim 37, wherein the precursor compounds processible by sol-gel processing include at least one metal alkoxide.

40. The composition of claim 39, wherein the metal alkoxide includes at least one of silicon alkoxides, tetraalkoxysilanes, oligomeric forms of tetraalkoxysilanes, alkylalkoxysilanes, aryltrialkoxysilanes, (meth)acrylsilanes, phenylsilanes, oligomeric silanes, polymeric silanes, epoxysilanes; fluoroalkylsilanes, fluoroalkyltrimethoxysilanes, or fluoroalkyltriethoxysilanes.

41. The composition of claim 40, further comprising at least one crosslinking agent including at least one of isocyanates, silanes, (meth)acrylates, 2-hydroxyethyl methacrylate, propyltrimethoxysilane, 3-(trimethylsilyl)propyl methacrylate, isophoron diisocyanate, hexamethylene-diisocyanate (HMDI), diethylenetriaminoisocyanate, 1,6-diisocyanatohexane, or glycerin.

42. The composition of claim 39, wherein the metal alkoxide includes at least one of a hydrolytically condensable, organically modified trialkoxysilane which contains free-radically polymerizable acrylate or methacrylate groups or cyclic groups capable of ring opening polymerization.

43. The composition of claim 1, wherein the composition is curable by drying, solvent extraction, radiation, such as visible light, UV or IR radiation, heat, polymerization or chemical crosslinking.

44. The composition of claim 1, further comprising at least one additive including at least one of a crosslinker, a silane coupling agent, a plasticizer, a solvent, a filler such as an inorganic filler such as silica powder, silver nanoparticles, quartz, glass beads, aluminum oxide, ceramics, salts, hydroxyl apatite; a stabilizer such as hydroquinone, hydroquinone monomethyl ether, t-butyl paracresol and hydroxy methoxybenzophenone, a pigment, or a beneficial agent.

45. The composition of claim 44, wherein the beneficial agent includes at least one of a pharmacologically, therapeutically, biologically or diagnostically active agent or an absorptive agent.

46. The composition of claim 45, wherein the beneficial ingredient is configured to be released in-vivo from the final implant.

47. The composition of claim 2, wherein the particles of metallic material comprise at least 5 wt.-% of the composition.

48. The composition of claim 2, wherein the particles of metallic material comprises from 1 to 99 wt.-% of the composition.

49. The composition of claim 2, wherein the particles of metallic material comprises from about 40 to 75 wt-% of the composition.

50. The composition of claim 1, wherein the organic material comprises at least about 5 wt.-% of the composition

51. The composition of claim 1, wherein the organic material comprises about 1 to 99 wt.-% of the composition.

52. The composition of claim 1, wherein the organic material comprises from about more preferred 10 to 80 wt.-% of the composition.

53. The composition of claim 1, wherein the organic material comprises from about 40 to 75 wt.-% of the composition.

54. The composition of claim 1, wherein the metallic material particles are modified with a coupling agent, preferably a silane coupling agent such as vinyl trichlorosilane, vinyl triethoxysilane, vinyl trimethoxysilane, vinyl tris(beta-methoxyethoxy)silane, and gamma-methacryloxypropyl trimethoxysilane.

55. A method of filling a cavity in a living organism, comprising:
providing an implant composition as follows
a) particles of a metallic material, and
b) a curable matrix-forming a non-particulate material,
wherein at least one of the metallic material or the matrix-forming material is at least partially degradable in-vivo;
filling the cavity with the implant composition in-vivo; and
curing the implant composition.

56. A method of filling a cavity in a living organism, comprising:
providing an implant composition providing an implant composition which includes:
a) particles of a metallic material, and
b) a curable matrix-forming a non-particulate material,
wherein at least one of the metallic material or the matrix-forming material is at least partially degradable in-vivo;
shaping the composition ex-vivo into a desired shape for filling the cavity;
curing the composition; and
implanting the cured composition into the cavity in the living organism.

57. The method of claim 55, wherein the cavity includes a defect or wound in a bone, or tooth or cartilage of a living organism.

58. The method of claim 56, wherein shaping is performed in a mold.

59. A use of an implant composition providing an implant composition, the composition comprising:
a) particles of a metallic material, and
b) a curable matrix-forming a non-particulate material,
wherein at least one of the metallic material or the matrix-forming material is at least partially degradable in-vivo, for repairing a defect or filling a cavity in a bone, tooth or cartilage in a living organism in-vivo.

60. A use of an implant composition, the composition comprising:
a) particles of a metallic material, and
b) a curable matrix-forming a non-particulate material,
wherein at least one of the metallic material or the matrix-forming material is at least partially degradable in-vivo, for repairing a defect or filling a cavity in a bone, tooth or cartilage in a living organism in-vivo for producing a shaped implant for repairing a defect or filling a cavity in a bone or cartilage in a living organism ex-vivo.

61. A use of an implant composition comprising a) particles of a metallic material, and b) a curable matrix-forming a non-particulate material,
wherein at least one of the metallic material or the matrix-forming material is at least partially degradable in-vivo, for repairing a defect or filling a cavity in a bone, tooth or cartilage in a living organism in-vivo, for producing a tissue scaffold, an implantable fracture fixation device such as plates, screws and rods, a dental implant, an orthopedic implant, a traumatologic implant, or a surgical implant.

62. A use of an implant composition, comprising
a) particles of a metallic material; and
b) a curable matrix-forming a non-particulate material,
wherein at least one of the metallic material or the matrix-forming material is at least partially degradable in-vivo, for repairing a defect or filling a cavity in a bone, tooth or cartilage in a living organism in-vivo, as a cement for fixation of implants or bone, or for repairing a bone fissure in a living organism in-vivo.
